SEQUENCE LISTING (10 sequences)

SEQ ID NO: 1 - DNA, 20 bp, synthetic construct
  source 1..20: mol_type = other DNA
tcctctctggctccatcgta

SEQ ID NO: 2 - DNA, 20 bp, synthetic construct
  source 1..20: mol_type = other DNA
ccgttctcctgtggattcgg

SEQ ID NO: 3 - DNA, 20 bp, synthetic construct
  source 1..20: mol_type = other DNA
gcaaaccttagaggttctgg

SEQ ID NO: 4 - RNA, 20 nt, synthetic construct
  source 1..20: mol_type = other RNA
accccacagtggggccacta

SEQ ID NO: 5 - DNA, 22 bp, synthetic construct
  source 1..22: mol_type = other DNA
ttctagggagcaggtcctgact

SEQ ID NO: 6 - DNA, 22 bp, synthetic construct
  source 1..22: mol_type = other DNA
tcctttcctacaagctcgtggg

SEQ ID NO: 7 - RNA, 20 nt, synthetic construct
  source 1..20: mol_type = other RNA
gatctccacataggggccag

SEQ ID NO: 8 - DNA, 81 bp, synthetic construct
  source 1..81: mol_type = other DNA
gggtacttttatctgtcccctccaccccacagtggggccaatagggacaggattggtgacagaaaagccccatccttaggc

SEQ ID NO: 9 - RNA, 20 nt, synthetic construct
  source 1..20: mol_type = other RNA
acggacccgcagacgctctc

SEQ ID NO: 10 - DNA, 100 bp, synthetic construct
  source 1..100: mol_type = other DNA
gcagaaggcagagcgtgagaaggagcggaggatggccaacaatgcccgagagcgtctgtgggtccgtgacatcaacgaggctttcaaagagctcggccgc

(19) United States
(12) Patent Application Publication
(10) Pub. No.: US 2024/0268371 A1
(43) Pub. Date: Aug. 15, 2024

(54) CRYOGENICALLY ARRAYABLE STABLE TRANSFECTION

(71) Applicant: Synthego Corporation, Redwood City, CA (US)

(72) Inventors: Bryant FLORES, Redwood City, CA (US); Ying PEI, Redwood City, CA (US); Alexander BROWN, Redwood City, CA (US)

(21) Appl. No.: 18/441,839

(22) Filed: Feb. 14, 2024

Related U.S. Application Data
(60) Provisional application No. 63/445,606, filed on Feb. 14, 2023.
(60) Provisional application No. 63/526,745, filed on Jul. 14, 2023.

(51) Int. Cl. A01N 1/02; C12N 5/074; C12N 9/22; C12N 15/11

(52) U.S. Cl. CPC A01N 1/0226; A01N 1/0231; C12N 5/0696; C12N 9/22; C12N 15/111; C12N 2310/20

(57) ABSTRACT

Provided herein systems and methods for processing cryopreserved cells without an expansion phase after thawing, including transfecting cells for gene editing purposes and cryopreserving transfected cells. Systems and methods for high-throughput processing of frozen cells are also provided. The systems and methods provided can be used to modify cells, including gene editing, in cell arrays.

### CROSS-REFERENCE TO RELATED APPLICATIONS

This application claims priority to U.S. Provisional Application No. 63/445,606 filed on Feb. 14, 2023, and U.S. Provisional Application No. 63/526,745 filed on Jul. 14, 2023, the contents of both of which are incorporated by reference in their entirety.

### REFERENCE TO AN ELECTRONIC SEQUENCE LISTING

The contents of the electronic sequence listing (174774.00168.xml; Size: 10,003 bytes; and Date of Creation: Feb. 13, 2024) is herein incorporated by reference in its entirety and is filed with this application.

### BACKGROUND

Transfection is a process widely used to introduce genetic material, such as nucleic acids (e.g., DNA or RNA) into cells. Transfection can be used to modify cells, for example by manipulating gene expression. There are numerous methods, workflows, and protocols for transfecting cells that are well known in the art.

Generally, the process of transfecting cells involves at least one to two weeks of preparing the cells for the transfection process. Cells are typically healthy, actively growing in an appropriate medium for a particular length of time to prepare them properly for efficient transfection.

A number of companies provide services for customers who order a modified cell generated using transfection processes. As a workflow, the company will receive an order, prepare the cells and the genetic material to be transferred into the cells, and contact the cells with the genetic material under the appropriate conditions to introduce the material into the cell and thereby modify the cell in the requested manner. For example, a gene can be regulated by introducing a CRISPR-Cas gene editing complex into the cell that targets the gene. CRISPR-Cas systems can be used to silence or activate gene expression. CRISPR stands for Clustered Regularly Interspaced Short Palindromic Repeats and has been well characterized over the past several years, including for potential therapeutic applications.

The rise in CRISPR and other gene editing activities over the years has increased the demand for modified and engineered cells generated using transfection processes. In addition, some cell lines are not viable after too many passages. There have been some efforts to speed up the process of manipulating cells on an individual level (i.e., one cell line at a time). For example, Wong et al. have described a process they call “CryoPause” specifically for use with human pluripotent stem cells (hPSCs) where the cells are cryopreserved in vials, thawed, washed, centrifuged, and then used in experiments, including genetically modifying the cells using transfection and CRISPR. Also, the company acCELLerate has described transfection ready cells that are cryopreserved. Once provided, the acCELLerate cells are seeded at 10,000 cells per well and grown over night to cultivate the cells before being transfected (www.accellerate.me/fileadmin/accellerate/Applications/Posters/TRC_Transfection_Ready_Cells_-_Poster.pdf). CCS Cell Culture Service GmbH described frozen instant cells that can be transfected or assayed after thawing and centrifuging to remove DMSO (dimethyl sulfoxide) (cdn.technologynetworks.com/ep/pdfs/instant-transient-transfection-of-bulk-frozen-cells.pdf).

Despite these processes, there remains a need to accelerate gene editing on multiple cells and multiple cell lines simultaneously without sacrificing quality, safety, and viability of the cells while maintaining a high degree of editing or transfection efficiency.

### SUMMARY

Disclosed herein are methods of processing frozen cells in an accelerated manner compared to traditional methods.

In certain aspects, the disclosed methods of processing cells comprise obtaining cells, wherein the cells are cryopreserved within a freezing media in a cell container; thawing the cells in the cell container; optionally transferring the thawed cells to a separate container; contacting the cells with genetic material and a cell manipulation solution to introduce the genetic material into the cells.

In some aspects, the freezing media is removed from the cell container after thawing the cells while not disturbing the cells pelleted at the bottom of the cell container, and the cells are resuspended before transferring them. The removal of the freezing media can be, for example, by aspiration. In other aspects, the freezing media is not removed after thawing.

In some aspects, the freezing media is serum-free and/or protein-free. In one aspect, the freezing media is serum and protein-free.

In some aspects, after cells are obtained and thawed, the cells are not subjected to centrifugation and are not washed before being contacted with the genetic material and/or the cell manipulation solution. In one aspect, cells frozen in serum-free media, protein-free media, or serum and protein-free media are not subjected to centrifugation and are not washed before being contacted with the genetic material and/or the cell manipulation solution. In some aspects the cells are not diluted or are not diluted more than 2, 3, 4, 5 or 10 fold between the thawing and contacting steps.

In some aspects, after cells are contacted with the genetic material and a cell manipulation solution, the cells are cryopreserved prior to allowing cell multiplication and/or prior to completion of gene editing. The cryopreserved cells may contain the Cas protein and the gRNA. The cryopreserved cells may be unedited, substantially unedited or only a small percentage of the cells may be edited, for example less than 2% or less than 1% edited. In certain aspects, the separate container is a well of a multi-well plate, such as a 96-well plate. In other aspects, the genetic material and cell manipulation solution contact the cells in the separate container. The separate container can optionally comprise the cell manipulation solution, the genetic material, or both the cell manipulation solution and the genetic material prior to the cells being transferred.

In one aspect, the genetic material can be introduced into the cells by electroporation.

In certain aspects, methods disclosed herein can be used for batch processing of cell editing events in a plurality of cell types and populations, wherein various cell types and cell populations are placed in prearranged positions in the separate cell container.

Also disclosed herein are methods of preparing cells for batch processing of cell editing transfections, the method comprising: expanding cells to form a cell bank; resuspending the cells in a freezing media; aliquoting the cells into a multiplicity of cell containers at a concentration of at least about 15,000 cells up to about 5×10⁷ cells per cell container, wherein each cell container has a volume of 1 mL or less; freezing the cells in the multiplicity of cell containers; and storing the cells for cell editing transfections. The containers making up the cell banks may each be used for one or more transfection. Each container in a cell bank may provide cells that can be used in 2, 3, 4, 5, 6 or more transfections so that the cells may be split into separate containers after thawing and be contacted with separate genetic material or separate amounts of genetic material. The separate genetic material may be the same or different.

Methods of high-throughput cell editing are also disclosed herein, the methods comprising: providing a plurality of arrayed cryopreserved cell populations; thawing the arrayed cell populations; and contacting the arrayed cell populations with genetic material and a cell manipulation solution to introduce the genetic material into the cells of the cell populations.

A composition comprising cryopreserved cells comprising a guide RNA and a Cas protein is also provided. In certain aspects, the cryopreserved cells have not been edited, or have not been substantially edited or have less than 5%, 3%, 2%, 1% of the cryopreserved cells edited by the Cas protein and gRNA.

Methods of using the composition comprising the cryopreserved cells are also disclosed herein. In certain aspects, the methods include thawing the cells and allowing the cells to be edited by the Cas protein and guide RNA. These thawed cells can also be allowed to multiply, clonally expanded, genotyped and selected for the targeted edit.

Methods of making cryopreserved transfected cells are also provided. The methods include contacting the cells with genetic material and a cell manipulation solution to introduce the genetic material into the cells. The genetic material may include reagents for CRISPR/Cas editing such as a guide RNA and a Cas protein or expression vector encoding a Cas protein. The genetic material may be introduced by electroporation or via other means of transfection. After introduction of the genetic material, the cells are cryopreserved. The cryopreservation step may be completed before allowing the cells to multiply or before allowing the expression of the genetic material by the cell. The cells may be cryopreserved before the guide RNA and Cas protein can edit, substantially edit or completely edit the cells.

### BRIEF DESCRIPTION OF THE FIGURES

FIG. 1 illustrates an automated liquid handler system set up with plates for transfecting cells. Cells from the Cell Source Plate can be transferred to the Mixing Plate along with ribonucleoprotein (RNP) complexes from the RNP Plate. The cells and RNP complexes can then be transferred to a Shuttle Plate, which is moved to a transfection system where transfection can be accomplished. Media from the media trough is added to the nucleofected cells, when are then transferred to empty Destination Plates.

FIG. 2A is a graph showing the editing efficiency results in IMR32 cells subjected to the Cryogenically Arrayable Stable Transfection (CAST) and standard transfection protocols.

FIG. 2B is a graph showing the editing efficiency results in HEPG2 cells subjected to the Cryogenically Arrayable Stable Transfection (CAST) and standard transfection protocols.

FIG. 2C is a graph showing the editing efficiency results in U2OS cells subjected to the Cryogenically Arrayable Stable Transfection (CAST) and standard transfection protocols.

FIG. 2D is a graph showing the editing efficiency results in PLC cells subjected to the Cryogenically Arrayable Stable Transfection (CAST) and standard transfection protocols.

FIG. 2E is a graph showing the editing efficiency results in HEK293 cells subjected to the Cryogenically Arrayable Stable Transfection (CAST) and standard transfection protocols.

FIG. 2F is a graph showing the editing efficiency results in A549 cells subjected to the Cryogenically Arrayable Stable Transfection (CAST) and standard transfection protocols.

FIG. 3 is a graph showing the editing efficiency results of cells subjected to the Cryogenically Arrayable Stable Transfection (CAST) system in various volumes of freezing media.

FIG. 4 is a graph showing the ability of the CAST system to successfully create double knock-out cells.

FIGS. 5A-5B are flow charts illustrating workflows for Cryogenically Arrayable Stable Transfection (CAST).

FIG. 6A are drawings illustrating workflows for delivery of standard transfected cells, Cryogenically Arrayable Stable Transfection (CAST) cells, and Cryogenically Arrayable Stable Transfection-Cryo ASap (CASTCas) cells.

FIG. 6B is a flowchart illustrating workflows for Cryogenically Arrayable Stable Transfection-Cryo Asap CASTCas.

FIGS. 7A-7B are graphs illustrating the ability of induced pluripotent stem cells (iPSC) to be to be genetically modified via A) Cryogenically Arrayable Stable Transfection (CAST) and B) a non-CAST transfection method. Indel percentages are represented by arrowheads, knock-ins percentages are represented by bars. Horizontal lines in B) represent historical averages for knock-ins (Kis).

FIG. 8 is a graph showing the viability of cells subjected to CAST and then immediately grown for 3 days in an incubator (“plated”) or immediately frozen and later thawed and then grown for 3 days (“thawed”).

FIG. 9 is a graph showing the editing efficiency results of cells from FIG. 8 determined by ICE.

FIG. 10 is a graph showing the viability of CASTCas cells at various timepoints after thawing.

FIG. 11 is a graph showing the percent of CASTCas cells edited by ICE at various timepoints after thawing.

FIG. 12 is a graph showing the effects of Knockout Serum Replacement (KSR), DMEM:F12, Dimethylsulfoxide (DMSO), and Freezing media (10% DMSO, 50% KSR, 40% DMEM:F12) on editing efficiency in cells undergoing the CAST process as measured by ICE.

FIG. 13 is a graph showing the effects of various components in freezing media (“cryo media”) on editing efficiency in cells undergoing the CAST process as measured by ICE.

FIG. 14 shows the effects of CAST on the pluripotency of various iPSC cell lines.

FIG. 15 shows the effects of CAST on genome stability in two different iPSC cell lines.

### DETAILED DESCRIPTION

Disclosed herein are methods referred to as Cryogenically Arrayable Stable Transfection (CAST), by which transfections are accomplished with cells without the time-consuming process of post-thaw expansion. In general, cells are received from a vendor and grown into banks (if needed) and stored indefinitely in cryogenic temperatures. These “cell banks” are taken from storage at cryogenic temperatures and replated in a process generally referred to as “thawing and plating.” This has been considered a necessary process for a successful transfection, as both the thawing and transfection process are known to be stressful events for the cell. Some methods of transfecting cells after thawing have been described (see Background above), but such methods still involve steps such as seeding and growing cells overnight and centrifuging and washing the cells to prepare them for further processing (e.g., transfections). As described herein, in CAST the thawing and plating steps are eliminated, as are centrifuging and washing steps. Instead, cells are taken directly from their cryogenic storage, thawed, and immediately taken into the transfection workflow as described herein.

The present invention is described herein using several definitions, as set forth below and throughout the application.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The present disclosure is directed to systems and methods for processing frozen cells without an expansion phase after thawing. More particularly, the systems and methods do not require centrifuging or washing after thawing. The present disclosure is also directed to systems and methods for high-throughput processing of frozen cells as described herein. Using disclosed methods, cells can be arrayed for batch cell editing events as described herein.

In some embodiments, the disclosed methods include obtaining cells. The cells are cryopreserved within a freezing media in a cell container and thawed in the cell container. The cell container can be from a cell bank prepared as described herein. Optionally at least a portion of the freezing media may be removed from the cell container after the cells are thawed. The portion of the freezing media removed from the cell container may be at least about 50%, 60%, 70%, 80%, 85%, 90%, or 95% of the freezing media in the container. The amount of freezing media removed from the cell container may be between 50-98%, between 55-95%, between 60-90%, between 65-85% or between 70-80% of the total freezing media in the cell container or any portion between any of these enumerated portions. Optionally the thawed cells are transferred to a separate container prior to contacting the cells with genetic material and a cell manipulation solution to introduce the genetic material into the cells. The separate container can be preloaded with genetic material (e.g., Cas protein and gRNA) or the genetic material (e.g., Cas protein and gRNA) can be added to the separate container of thawed cells, and after mixing with the genetic material the cells may be transferred to an electroporation cuvette or vial suitable for electroporation for the introduction of genetic material into the thawed cells. The cells may be transferred from a freezing vial to an electroporation cuvette or vial suitable for electroporation for the contacting and introduction of genetic material into the thawed cells. In some embodiments, the freezing media and electroporation buffer are both present in the electroporation cuvette or vial suitable for electroporation. The freezing media may be serum-free media, protein-free media, or serum and protein-free media. Obtaining cells may include receiving cells from a cell bank or other cell source. For example, the cells may be obtained from a local freezer or liquid nitrogen storage system. In another example, the cells may arrive frozen (e.g., packed in dry ice) from a hospital, clinic, laboratory or cell repository. In some embodiments, a container of cells from a cell bank has a concentration of at least about 15,000 cells up to about 5×10⁷ cells per cell container (e.g., in 100 uL), wherein each cell container has a volume of 1 mL or less.

Cryopreserved cells can be provided by any means of freezing cells in a cryopreservation media as known in the art. For example, cells can be grown to desired number and aliquoted in cryogenic storage vials and frozen at a controlled rate in a freezing apparatus as described, for example, at www.thermofisher.com/us/en/home/references/gibco-cell-culture-basics/cell-culture-protocols/freezing-cells.html. Any cryopreservation (freezing) media can be used according to the particular cell type and other factors known to those of skill in the art. For example, Recovery™ Cell Culture Freezing Medium (Thermo Fisher Scientific Inc.), M-FREEZE™ Cryoembedding media and CryoStor® cell cryopreservation media (MilliporeSigma), and media from vendors such as R&D Systems, BIO-RAD, BPS Bioscience, and others.

As used herein, the terms “frozen” and “cryopreserved” are used interchangeably. The terms “freezing media” and “cryopreservation media” are also used interchangeably.

Thawed cells can be placed in any suitable container for the desired manipulation step as will be appreciated by those skilled in the art. For example, the cells can be placed into wells of a multi-well plate (e.g., a 96 well plate). In some embodiments, a manipulation step can also be accomplished after thawing within the cell container in which the cells were frozen.

A “cell container” can be a cryogenic freezing vial, which is well known and readily available to those of skill in the art. Preferably, the container is a vial that can be placed into a well of a 96 well plate or other such holder. The cell container may also include a plate containing multiple wells (e.g., 6, 12, 24, 48, 96, 384, or 1536 wells). For example, the cell container may include 2 mL cryogenic vials manufactured by the Nalgene company. The Micronics 96 well matrix tubes or Azenta 96 well 0.5 mL tubes may also be used. In some embodiments, the cells are cryopreserved in cell containers, or wells of containers, that hold less than 10 mL, less than 5 mL, less than 2 mL, less than 1.5 mL, less than 1.0 mL, less than 0.5 mL, less than 0.25 mL, less than 0.2 mL, less than 0.1 mL, less than 0.05 mL, less than 0.01 mL. For example, the containers hold between 0.01 mL to 1.5 mL. In another example, the volume of the containers may range from 0.25 mL to 0.5 mL.

In some embodiments, the containers are 1.5 mL tubes/vials that can be capped and are suitable for automation. For example, an Azenta 96 well capper/decapper systems (Azenta Life Sciences) can be used to cap and uncap up to 96 tubes/vials of frozen cells in any combination. The tubes can also be barcoded or otherwise marked for case of sample identity to process many cell samples at the same time.

Cells are cryopreserved in the vials at a concentration of greater than 1,000 cells/mL, greater than 10,000 cells/mL, greater than 100,000 cells/mL, greater than 150,000 cells/mL, greater than 1e6 cells/mL, greater than 1.5e6 cells/mL, greater than 2e6 cells/ml, greater than 10e6 cells/ml, greater than 20e6 cells/ml, greater than 10e7 cells/mL, or greater than 20e7 cells/mL. In particular examples, the vials hold 1e6 cells/100 μL. The cells may also be cryopreserved in a range of 15,000 cells/100 μL to 20e6 cells/100 μL, of 100,000 cells/100 μL to 10e6 cells/100 μL, or 500,000 cells/100 μL to 5e6 cells/100 μl.

In some embodiments, the cell manipulating step after thawing comprises introducing genetic material into the cell. This step occurs immediately after thawing the cells. “Immediately” as used herein means less than about 60, 50, 45, 40, 35, 30, 25, 20, 15, 10, 7, 5, 3, 2, 1, or 0.5 minutes after completion of thawing.

The manipulation step can comprise any means of transferring genetic material into the cell. In one embodiment, the transferring is accomplished by transfection, including but not limited to electroporation (including transfection), lipid-mediated transfection, biolistic particle delivery, and viral vector mediated delivery.

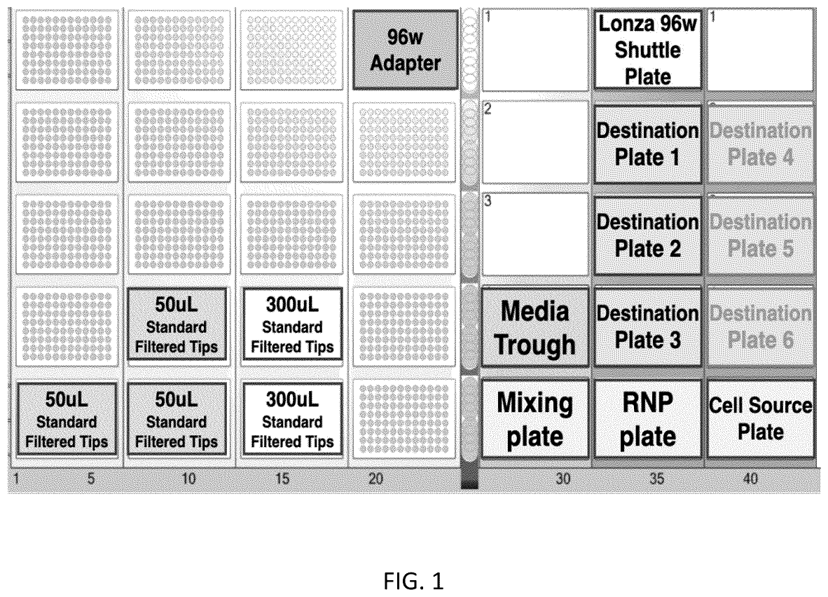

In some embodiments, the cells are thawed, and the method follows in an automated manner without being handled by a human. For example, one or more steps may be carried out by a robotic liquid handling system. The robotic system may include one or more controllers that control movements of one or more robotic elements of the robotic system. For example, the robotic system may include one or more robotic arms or gantries controlled by the one or more controllers that can grab or latch onto the cell container and move the cell container to the appropriate position. In another example, the robotic system may include one or more liquid dispensers controlled by the one or more controllers that can add a liquid to an appropriate cell container, resuspend cells, wash cells, or transfer cells (e.g., thawed cells) from one container to another separate container. The robotic system may include one or more processors (e.g., circuits, microcontrollers, or central processing units) operatively coupled to the one or more controllers. The robotic system may further include memory (e.g., volatile or nonvolatile) operatively coupled to the one or more processors and configured to store instructions that, when executed by the one or more processors, cause the controller to operate the robotic elements (e.g., the one or more robotic arms and/or the one or more liquid dispensers). For instance, the method can be carried out by a robotic system configured as an automated liquid handler as shown in FIG. 1, such as those provided by Hamilton Company and as described in Example 1.

Thawing can occur at room temperature, or in heated water bath (e.g., 37° C.), or in a thawing chamber (e.g., an incubator optionally including a fan to increase air flow around the vials) having a temperature (e.g., about 25° C. to about 37° C.) to control the rate of thawing. The thawing is allowed to occur over a period of less than about 10, 9, 8, 7, 6, 5, 4, 3, 2, 1, or 0.5 minutes.

In some embodiments, the freezing media is removed from the cell container after thawing the cells but is done so without disturbing the cells pelleted at the bottom of the cell container (e.g., preventing cells from releasing from the pellet and being removed along with the freezing media). When the freezing media is removed, the cells are resuspended and then optionally transferred to a separate container. Preferably, when removing the freezing media before transfer, at least 50%, 60%, 70%, 80%, 90%, or 95% of the freezing media is removed from the cell container after thawing and before resuspending the cells and optionally transferring to the separate cell container. The removal of the freezing media can be by aspiration. Cells naturally fall to the bottom of their container during the cryopreservation process. Thus, the media can be aspirated via vacuum line, centrifugal pump, pipette, or other means known to those of skill in the art at a specific height/remaining volume so as not disturb the cell pellet. In some instances, the media may be decanted if a cell pellet is formed that is adhered to the container.

The freezing media may include any liquid solution that aids in the cryopreservation process. Commercial freezing medias include but are not limited to CRYOSTOR CS10 freeze media (Biolife Solutions), Cell Freezing Media (Thermo Fisher Scientific), RECOVERY Cell Culture Freezing Medium (Thermo Fisher Scientific), and Cell Culture Freezing Medium (Cell Biologics). Freezing medias may also include commonly used cell media solutions (e.g., DMEM or RPMI1640) containing 5% to 30% serum (e.g., calf serum or fetal bovine serum) and 5% to 30% dimethyl sulfoxide (DMSO). For example, the freeing media may include 10% fetal bovine serum and 10% DMSO. In another example, the freezing media may include 90% serum and 10% DMSO. The freezing media may include a mixture of different freezing medias or a mixture of a freezing media with growth media. In some embodiments, the freezing media is serum-free and/or protein-free. In certain embodiments, the freezing media is serum and protein-free.

In other embodiments, the freezing media is not removed from the cell container after thawing the cells. The cells with the freezing media can be transferred to the separate container at this stage or can remain in the cell container for further steps. The cells or cell pellet may also be resuspended at this point, or at another point within the method. For example, a cell pellet may be resuspended by repeatedly drawing cells or cell pellet through a pipette (e.g., manually or via an automated liquid handler). The cells may not be diluted at this point or may be diluted 2, 3, 4, 5, 6, 7, 8, or even 10-fold. The dilution and resuspension of the cells may include the cell manipulation solution and the genetic material. This step may prepare the cells for the contact with the genetic material and introduction of the genetic material into the cells. The separate container may be an electroporation cuvette or vial suitable for electroporation and the volume added to the separate container may be as little as 25 μL and up to 1 mL or any value in between, such as 30 μL, 50 μL, 100 μL, 200 μL, 500 μL or values in between these volumes. The number of cells added to the separate containers may be between 1e4 and 1e7. In some aspects, the number of cells added to the separate container may be between 1e6 and 1e7. The separate container has a total volume of less than 5 mL, less than 2 mL, less than 1 mL, less than 0.5 mL. The volume of the separate container may be greater than 50 μL, greater than 100 μL, greater than 200 μL or any volume between those presented here. In some embodiments, two separate containers are used, one in which the thawed cells and genetic material are mixed together along with a cell manipulation solution (e.g., an electroporation buffer) and a second that is an electroporation cuvette or vial suitable for electroporation.

In some embodiments, the freezing media is serum-free and/or protein-free and is not removed from the cell container after thawing the cells, the media and the thawed cells are then transferred to a container that has a manipulation solution and genetic material after thawing. In other embodiments, the manipulation solution and genetic material are added to the thawed cells before being transferred to a separate container for transfection (e.g., electroporation). There may be no need to transfer the cells to a separate container if the cell container used for cryopreservation is also suitable for the transfection methods being used with the cells.

The separate container can be the well of a multi-well plate. The multi-well plate can be a 4-well plate, a 6-well plate, a 12-well plate, a 24-well plate, a 48-well plate, a 96-well plate, or a 384-well plate. The separate container can already include a cell manipulation solution, genetic material to be transferred to the cell, or both. Alternatively, the cell manipulation solution and genetic material can be added to the separate container after the cells.

In some embodiments, the cells can be allowed to remain in the cell container and the manipulation solution with genetic material can be added directly to the cells in the cell container.

Once the cell manipulation solution and genetic material and cells are together, they are subjected to appropriate conditions to enable transfection according to the selected transfection method, including to an electroporation device to introduce the genetic material into the cells.

The cell manipulation solution may include any type of solution (salt/buffer solutions, transfection buffer/solutions, transfection buffer/solution, electroporation buffer/solution) that keeps the cells viable during the manipulation step. For example, salt/buffer solutions may include but not be limited to phosphate buffered saline (PBS), Dulbecco's Phosphate-Buffered Saline (DPBS), Hanks' Balanced Salt Solution (HBSS), or HEPES buffer solution. Recipes for transfection, Nucleofection®, and electroporation solutions may be hand-crafted or produced commercially. For example, the Lonza company produces a proprietary nucleofection buffer, Solution V, for use in their nucleofection kits. In another example, the transfection solution may contain one of many proprietary lipid-based compositions that are commercially available.

In some embodiments, the genetic material includes one or more polynucleotides, such as a guide RNA, and a nuclease protein for CRISPR applications to be used for a desired gene editing event. In certain embodiments, the “genetic material” comprises polynucleotides or a construct comprising nucleic acid molecules. The constructs may be plasmid or viral vectors and the nucleic acid molecules may be operably connected to promoters to allow for expression of the molecules. Genetic material may be components of an endonuclease-based gene editing system, such as a guide RNA (gRNA) and/or a nuclease protein for CRISPR applications (e.g., CRISPR ribonucleoprotein complexes) to be used for a desired gene editing event.

### Genetic Material and Gene Editing

Gene editing is well known in the art to modify cells. The methods provided herein may be used to prepare genetically modified cells via gene editing.

The genetically modified cells may be prepared using recombination methods known in the art. In some embodiments, the genetically modified cells are prepared using homologous recombination methods (e.g., microhomology-mediated end joining or homology directed repair). In other embodiments, the genetically modified cells are prepared using non-homologous recombination methods (non-homologous end joining).

The genetically modified cells may be prepared by recombination methods that utilize nucleases to promote recombination at selected genomic sites (e.g., as effector proteins). Suitable nucleases may include clustered repeat interspaced short palindromic repeats (CRISPR) effector polypeptides, for example, a type II CRISPR effector polypeptide such as a Cas9 polypeptide and type V CRISPR effector polypeptides such as a Cas12a, a Cas12b, a Cas12c, a Cas12d, a Cas12c, a Cas12f, a Cas12g, a Cas12h or a Cas12i polypeptide). Suitable CRISPR-effector polypeptides also may include a Cas14a, a Cas14b, or a Cas14c polypeptide.

Suitable nucleases for preparing the genetically modified cells may include non-CRISPR effector polypeptides. Other suitable nucleases may include zinc finger nucleases (ZFNs) and transcription activator-like effector nucleases (TALENs).

In some embodiments, the genetically modified cells may be prepared by a method comprising introducing into a cell: (a) a CRISPR effector protein or a polynucleotide encoding a CRISPR effector protein; (b) a guide polynucleotide comprising a guide sequence designed to hybridize with a target sequence in the cell; and optionally (c) a donor polynucleotide comprising an exogenous polynucleotide sequence. For example, the method may include the CRISPR effector protein introducing a double-stranded break at the target sequence with repair of the double-stranded break occurring through a DNA repair process that results in a deletion in the gene or an insertion of the inserted polynucleotide sequence into the genetic target within the cell thereby producing a modified cell with the intended genetic change. In some embodiments, the inserted polynucleotide sequence may encode a protein that is inserted in-frame with the coding sequence of the native gene such that the genetically modified gene encodes a novel fusion protein. In other embodiments, the inserted polynucleotide sequence knocks out an endogenous gene.

The term “polynucleotide” or “nucleic acid,” as used interchangeably herein, can generally refer to a polymeric form of nucleotides of any length, either ribonucleotides and/or deoxyribonucleotides. Thus, these terms include, but are not limited to, single-, double-, or multi-stranded DNA or RNA, genomic DNA, complementary DNA (cDNA), guide RNA (gRNA), messenger RNA (mRNA), DNA-RNA hybrids, or a polymer comprising purine and pyrimidine bases or other natural, chemically or biochemically modified, non-natural, or derivatized nucleotide bases. The term “oligonucleotide,” as used herein, can generally refer to a polynucleotide of between about 5 and about 100 nucleotides of single- or double-stranded DNA or RNA. However, for the purposes of this disclosure, there may be no upper limit to the length of an oligonucleotide. In some cases, oligonucleotides can be known as “oligomers” or “oligos” and can be isolated from genes, or chemically synthesized by methods known in the art. The terms “polynucleotide” and “nucleic acid” should be understood to include single-stranded (such as sense or antisense) and double-stranded polynucleotides.

The term “modified nucleotide,” as used herein, can generally refer to a nucleotide having a modification to the chemical structure of one or more of the base, the sugar, and/or the phosphodiester linkage or backbone portions, including the nucleotide phosphates, relative to a naturally occurring base, sugar, and/or phosphodiester linkage or backbone portions.

The term “hybridization” or “hybridizing,” as used herein, can generally refer to a process where completely or partially complementary polynucleotide strands come together under suitable hybridization conditions to form a double-stranded structure or region in which the two constituent strands are joined by hydrogen bonds. In some cases, modified nucleotides can form hydrogen bonds that allow or promote hybridization. In some cases, a guanine (G) of a protein-binding segment of a subject DNA-targeting RNA molecule can be considered complementary to a uracil (U), and vice versa.

The term “CRISPR/Cas,” as used herein, refers to a ribonucleoprotein (“RNP”) complex comprising a guide RNA (gRNA) and a CRISPR-associated (Cas) endonuclease. The term “CRISPR” refers to the Clustered Regularly Interspaced Short Palindromic Repeats and the related system thereof. While CRISPR was discovered as an adaptive defense system that enables bacteria and archaea to detect and silence foreign nucleic acids (e.g., from viruses or plasmids), it can be adapted for use in a variety of cell types to allow for polynucleotide editing in a sequence-specific manner. In some cases, one or more elements of a CRISPR system can be derived from a type I, type II, or type III CRISPR system. In the CRISPR type II system, the guide RNA can interact with Cas and direct the nuclease activity of the Cas enzyme to a target region. The target region can comprise a “protospacer” and a “protospacer adjacent motif” (PAM), and both domains can be needed for a Cas enzyme mediated activity (e.g., cleavage). The protospacer can be referred to as a target site (or a genomic target site). The gRNA can pair with (or hybridize) the opposite strand of the protospacer (binding site) to direct the Cas enzyme to the target region. The PAM site generally refers to a short sequence recognized by the Cas enzyme and, in some cases, required for the Cas enzyme activity. The sequence and number of nucleotides for the PAM site can differ depending on the type of the Cas enzyme.

The term “Cas,” as used herein, can generally refer to a wild type Cas protein, a fragment thereof, or a mutant or variant thereof. A Cas protein can comprise a protein of or derived from a CRISPR/Cas type I, type II, or type III system, which can be an RNA-guided polynucleotide-binding or nuclease activity. Examples of suitable Cas proteins include CasX, Cas3, Cas4, Cas5, Cas5c (or CasD), Cas6, Cas6e, Cas6f, Cas7, Cas8a1, Cas8a2, Cas8b, Cas8c, Cas9 (also known as Csn1 and Csx12), Cas10, Cas10d, CasF, CasG, CasH, Csy1, Csy2, Csy3, Cse1 (or CasA), Cse2 (or CasB), Csc3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, homologues thereof, and modified versions thereof. In some cases, a Cas protein can comprise a protein of or derived from a CRISPR/Cas type V or type VI system, such as Cpf1 (Cas12a), C2c1 (Cas12b), C2c2, homologues thereof, and modified versions thereof. In some cases, a Cas protein can be a catalytically dead or inactive Cas (dCas).

The term “guide RNA” or “gRNA,” as used herein, can generally refer to an RNA molecule (or a group of RNA molecules collectively) that can bind to a Cas protein and aid in targeting the Cas protein to a specific location within a target polynucleotide (e.g., a DNA). A guide RNA can comprise a CRISPR RNA (crRNA) segment and a trans-activating crRNA (tracrRNA) segment. The term “crRNA” or “crRNA segment,” as used herein, can refer to an RNA molecule or portion thereof that includes a polynucleotide-targeting guide sequence, a stem sequence, and, optionally, a 5′-overhang sequence. The term “tracrRNA” or “tracrRNA segment,” can refer to an RNA molecule or portion thereof that includes a protein-binding segment (e.g., the protein-binding segment is capable of interacting with a CRISPR-associated protein, such as a Cas9). The term “guide RNA” can encompasse a single guide RNA (sgRNA), where the crRNA segment and the tracrRNA segment are located in the same RNA molecule. The term “guide RNA” can also encompasse, collectively, a group of two or more RNA molecules, where the crRNA segment and the tracrRNA segment are located in separate RNA molecules.

In some cases, the CRISPR/Cas activity can be useful to modify a nucleic acid in a cell in a site-specific (targeted) way, for example gene knock-out (KO), gene knock-in (KI), gene editing, gene tagging, etc., as used in, for example, gene therapy. The nucleic acid can be DNA or RNA. Examples of gene therapy include treating a disease or as an antiviral, antipathogenic, or anticancer therapeutic; the production of genetically modified organisms in agriculture; the large-scale production of proteins by cells for therapeutic, diagnostic, or research purposes; the induction of induced pluripotent stem cells (iPS cells or iPSCs); and the targeting of genes of pathogens for deletion or replacement. In some cases, the Cas can be a catalytically dead or inactive Cas (dCas), and the resulting CRISPR/dCas system can be useful for sequence-specific repression (CRISPR interference) or activation (CRISPR activation) of gene expression.

The term “subject,” “individual,” or “patient,” as used herein, can generally refer to whole organism or a collection thereof that can be in need of and/or subjected to a treatment, such as a farm animal, companion animal, or human, or a collection thereof. In some cases, the term “subject” can be a cell or a cell line thereof.

As used herein, a subject in need thereof may include a subject having or at risk for developing a disease or disorder that may be treated and/or prevented by modulating an immune response in the subject. As disclosed herein, “modulation” may include induction and/or enhancement of an immune response in a subject. As disclosed herein, “modulation” also may include reduction or elimination of an immune response and/or induction of tolerance in a subject. A subject may include a human subject or a non-human subject (e.g., dogs, cats, horses, cows, pigs, and the like).

The term “gene,” as used herein, can generally refer to a nucleotide sequence that encodes functional genetic information, such as for example, a nucleotide sequence encoding a polypeptide (e.g., protein), a transfer RNA (tRNA), or a ribosomal RNA (rRNA). The gene can comprise DNA, RNA, or other nucleotides.

Designing gRNA Oligonucleotides

In an aspect, the present disclosure discloses methods for transfecting cells with one or more guide RNAs (gRNAs) for hybridizing to a genomic region of interest in a cell. The genomic region of interest can be a gene of a genome of a species. The gRNA oligonucleotides can be generated by selecting a transcript from a plurality of transcripts of the gene. The method can comprise identifying an initial gRNA or set of gRNAs that hybridize different target sites in the gene of the selected transcript. The gene can be a gene of interest. The genomic region of interest can be a non-coding region of the genome. The non-coding region can be a regulatory element. The regulatory element can be a cis-regulatory element or a trans-regulatory element. The cis-regulatory element can be a promoter, an enhancer, or a silencer.

Information comprising the genome of the species and/or a reference genome of the species can be obtained from a plurality of databases. In some cases, the plurality of databases can include gene and/or genome databases comprising sequencing data from DNA (DNA-seq) and/or RNA (RNA-seq). Examples of such genome databases include GENCODE, NCBI, Ensembl, {APPRIS}, and NIH Human Microbiome Project. Alternatively, or in addition to, genomic information of an individual can be retrieved from personalized genome databases, including, but are not limited to, 23andMe, deCODE Genetics, Gene by Gene, Gene Planet, DNA Ancestry, uBiome, and healthcare providers. In some cases, necessary information comprising at least a portion of the genome of the species of interest can be provided by a user (e.g., via a user interface on a user device such as a personal computer).

The genome of the species can comprise some or a complete set of genetic material present in the species (e.g., cell or organism). Examples of the species include, but are not limited to, mammals (e.g., Homo sapiens, Mus musculus, Cricetulus griseus, Rattus norvegecus, Pan paniscus), fish (e.g., Danio rerio, Amphiprion frenatus), insect (e.g., Drosophila melanogaster), plants (e.g., Arabidopsis thaliana), roundworms (e.g., Caenorhabditis elegans), and microorganisms including bacteria (e.g., Escherichia coli, Lactobacillus bulgaricus). In some cases, the bacteria can include strains that are to be consumed by an individual as a supplement (e.g., in yogurt as a medium) and/or as a treatment (e.g., to suppress or ameliorate a condition). In some cases, the bacteria can include strains that are present in the body of an individual (e.g., human microbiome).

The genetic material of the genome can be DNA and/or RNA. The genetic material can include nucleic acid sequences in genes and intergenetic regions. In some cases, the genetic material can be represented as a unit of a chromosome. In some cases, the genetic material can be represented as one or more transcripts that have been transcribed from a gene. The gene and its respective one or more transcripts can comprise one or more coding regions (i.e., exons). In some cases, the gene and its respective one or more transcripts can comprise one or more intragenic non-coding regions (i.e., introns). The one or more intragenic non-coding regions can be located between the coding regions. In some cases, a gene can encode one transcript. In some cases, a gene encodes a plurality of transcripts, each transcript comprising different variations of exons and introns from the gene. In an example, the RelA gene encodes for transcription factor p65, and the RELA gene of Homo sapiens encodes at least 18 known transcripts of varied length: RELA-202, RELA-207, RELA-226, RELA-205, RELA-201, RELA-208, RELA-220, RELA-207, RELA-215, RELA-204, RELA-222, RELA-213, RELA-225, RELA-211, RELA-219, RELA-221, and RELA-212. Thus, the plurality of transcripts can have different numbers of nucleotide bases (polynucleotide lengths). Alternatively or in addition to, the plurality of transcripts can be translated into polypeptides (e.g., proteins) having different numbers of amino acids (polypeptide lengths). In some cases, each of the plurality of transcripts can have different expression levels (abundance) reported relative to one or more other transcripts.

The gRNA or set of gRNAs can be designed to hybridize to a target region, also referred to herein as a binding site. The target region can be in a gene or a portion of the gene in the genome of the species. In some cases, the portion of the gene can be an exon of the gene. The exon can be an exon found in each transcript of the gene. The exon can be the selected exon of a selected transcript of the plurality of transcripts of the gene from the aforementioned criteria. In some cases, one or more gRNAs in the initial set of gRNAs can be a single guide RNA (sgRNA). In some cases, the sgRNA can be a single polynucleotide chain. The sgRNA can comprise a hybridizing polynucleotide sequence and a second polynucleotide sequence.

The hybridizing polynucleotide sequence can hybridize to the portion of the gene (e.g., the selected exon of the selected transcript of the plurality of transcripts of the gene). The hybridizing polynucleotide sequence of the sgRNA can range between 17 to 23 nucleotides. The hybridizing polynucleotide sequence of the sgRNA can be at least 17, 18, 19, 20, 21, 22, 23, or more nucleotides. The hybridizing polynucleotide sequence of the sgRNA can be at most 23, 22, 21, 20, 19, 18, 17, or less nucleotides. In an example, the hybridizing polynucleotide sequence of the gRNA is 20 nucleotides. The hybridizing polynucleotide sequence can be complementary or partially complementary to the target region. A hybridizing polynucleotide sequence complementary to the target region can comprise a sequence with 100% complementarity to a sequence of the target region. A gRNA partially complementary to the target region can comprise a sequence with at least 1, at least 2, at least 3, at least 4, or at least 5 mismatches relative to a sequence comprising 100% complementary to the target region.

A second polynucleotide sequence of the single polynucleotide chain sgRNA can interact (bind) with the Cas enzyme. The second polynucleotide sequence can be about 80 nucleotides. The second polynucleotide sequence can be 80 nucleotides. The second polynucleotide sequence can be at least 80, or more nucleotides. The second polynucleotide sequence can be at most 80, or less nucleotides.

Overall, the single polynucleotide chain sgRNA can range between 97 to 103 nucleotides. The single polynucleotide chain sgRNA can be at least 97, 98, 99, 100, 101, 102, 103, or more nucleotides. The single polynucleotide chain sgRNA can be at most 103, 102, 101, 100, 99, 98, 97, or less nucleotides. In an example, the single polynucleotide chain sgRNA can be 100 nucleotides.

In some cases, gRNA or one or more gRNAs in a set of gRNAs can be a complex (e.g., via hydrogen bonds) of a CRISPR RNA (crRNA) segment and a trans-activating crRNA (tracrRNA) segment. The crRNA can comprise a hybridizing polynucleotide sequence and a tracrRNA-binding polynucleotide sequence. The hybridizing polynucleotide sequence can hybridize to the portion of the gene (e.g., the selected exon of the selected transcript of the plurality of transcripts of the gene). The hybridizing polynucleotide sequence of the crRNA can range from 17 to 23 nucleotides. The hybridizing polynucleotide sequence of the crRNA can be at least 17, 18, 19, 20, 21, 22, 23, or more nucleotides. The hybridizing polynucleotide sequence of the crRNA can be at most 23, 22, 21, 20, 19, 18, 17, or less nucleotides. In an example, the hybridizing polynucleotide sequence of the crRNA is 20 nucleotides. The tracrRNA-binding polynucleotide sequence of the crRNA can be 22 nucleotides. The tracrRNA-binding polynucleotide sequence of the crRNA can be at least 22, or more nucleotides. The tracrRNA-binding polynucleotide sequence of the crRNA can be at most 22, or less nucleotides. Overall, the crRNA can range from 39 to 45 nucleotides. The crRNA can be at least 39, 40, 41, 42, 43, 44, 45, or more nucleotides. The crRNA can be at most 45, 44, 43, 42, 41, 40, 39, or less nucleotides. The tracrRNA can range from 60 and 80 nucleotides. The tracrRNA can be at least 60, 61, 62, 63, 64, 66, 68, 70, 72, 74, 76, 78, 80, or more nucleotides. The tracrRNA can be at most 80, 79, 78, 77, 76, 74, 72, 70, 68, 66, 64, 62, 60, or less nucleotides. In an example, the tracrRNA can be 72 nucleotides. In another example, the hybridizing polynucleotide sequence of the crRNA is 20 nucleotides, the crRNA is 42 nucleotides, and the respective tracrRNA is 72 nucleotides.

In some cases, a gRNA or set of gRNAs can comprise both one or more sgRNAs and one or more complexes of the crRNA and the tracrRNA. Alternatively, or in addition to, one or more gRNAs in the initial set of gRNAs can be a complex of three or more RNA chains. At least one RNA chain of the complex of three or more RNA chains can comprise a hybridizing polynucleotide sequence. At least one RNA chain of the complex of three or more RNA chains can comprise a Cas enzyme binding sequence.

### Genetically Modified Cells

Disclosed are methods for preparing genetically modified cells for use in research and development activities, including preclinical and clinical development.

The genetically modified cells can be prepared and frozen for a future therapeutic application. Thus, the CAST process can be applied to generate modified cells, the cells are frozen and stored until a patient needs the cells for a therapeutic application. In some embodiments, the cells are primary cells obtained from the patient or primary cells obtained from a donor. Primary cells can be cryopreserved and later modified using methods described herein. In other embodiments, the cryopreserved cells can be modified and banked or stored for future uses.

In some embodiments, methods disclosed herein can be applied in a cell therapy procedure, wherein the CAST process is applied to frozen cells that are provided for a particular patient. The cells are thawed at the site of treatment for the patient and the modified cells are then administered to the patient for treatment. In some embodiments, there is a subsequent CAST process applied before giving the modified cell to the patient, thus a second (or third) cell manipulation event can be conducted before the cells are administered to the patient.

In the disclosed methods for preparing genetically modified cells, suitable donor polynucleotides may include single stranded DNA and/or double stranded DNA. Vectors may be utilized in order to provide donor polynucleotides in the disclosed methods. Suitable vectors may include viral vectors, plasmids, and transposons.

Methods disclosed herein can be used to manipulate/modify a variety of cells. Cells can be any prokaryotic or eukaryotic living cells, cell lines derived from these organisms for in vitro cultures, primary cells from animal or plant origin. Eukaryotic cells can refer to a fungal, plant, algal or animal cell or a cell line derived from the organisms listed below and established for in vitro culture. The fungus can be of the genus Aspergillus, Penicillium, Acremonium, Trichoderma, Chrysoporium, Mortierella, Kluyveromyces or Pichia; More preferably, the fungus is of the species Aspergillus niger, Aspergillus nidulans, Aspergillus oryzae, Aspergillus terreus, Penicillium chrysogenum, Penicillium citrinum, Acremonium Chrysogenum, Trichoderma reesei, Mortierella alpine, Chrysosporium lucknowense, Kluyveromyceslactis, Pichia pastoris or Pichia ciferrii. The plant can be of the genus Arabidospis, Nicotiana, Solanum, lactuca, Brassica, Oryza, Asparagus, Pisum, Medicago, Zea, Hordeum, Secale, Triticum, Capsicum, Cucumis, Cucurbita, Citrullis, Citrus, Sorghum. The plant can be of the species Arabidospis thaliana, Nicotiana tabaccum, Solanum lycopersicum, Solanum tuberosum, Solanum melongena, Solanum esculentum, Lactuca saliva, Brassica napus, Brassica oleracea, Brassica rapa, Oryza glaberrima, Oryza sativa, Asparagus officinalis, Pisumsativum, Medicago sativa, Zea mays, Hordeum vulgare, Secale cereal, Triticuma estivum, Triticum durum, Capsicum sativus, Cucurbitapepo, Citrullus lanatus, Cucumis melo, Citrus aurantifolia, Citrus maxima, Citrus medica, and Citrus reticulata. The animal cell can be of the genus Homo, Rattus, Mus, Cricetulus, Pan, Sus, Bos, Danio, Canis, Felis, Equus, Salmo, Oncorhynchus, Gallus, Meleagris, Drosophila, Caenorhabditis. The animal cell can be of the species Homo sapiens, Rattus norvegicus, Mus musculus, Cricetulus griseus, Pan paniscus, Sus scrofa, Bos taurus, Canis lupus, Cricetulus griseus, Danio rerio, Felis catus, Equus caballus, Rattus norvegecus, Salmo salar, Oncorhynchus mykiss, Gallus, Meleagris gallopavo, Drosophila melanogaster, and Caenorhabditis elegans.

Examples of cell lines can be selected from the group consisting of CHO cells (e.g., CHO-K1); HEK293 cells; Caco2 cells; U2-OS cells; NIH 3T3 cells; NSO cells; SP2 cells; DG44 cells; K-562 cells, U-937 cells; MC5 cells; IMR90 cells; Jurkat cells; HepG2 cells; HeLa cells; HT-1080 cells; HCT-116 cells; Hu-h7 cells; Huvec cells; and Molt 4 cells. Examples of other cells applicable to the scope of the present disclosure can include primary cells, stem cells, human pluripotent stem cells (hPSCs), embryonic stem cells (ESCs), induced pluripotent stem cells (iPSCs), white blood cells, lymphocytes (e.g., B-cells, T-cells, NK cells), monocytes, leucocytes (e.g., microglia, dendritic cells), and T-cells engineered with chimeric antigen receptors (e.g., CAR T cells). In some embodiments, the cell or cell lines are adherent cells, including for example: A549, HEK293, U2OS, HEPG2, A375, CT26. WT, HCT116, HELA, PANCI, IMR32, MCF7, PLCPRF5, Mia-Paca2, and HEK293T cells. In other embodiments, the cell or cell lines are suspension cells, including for example: THP1, Jurkat, K562, RPMI8226, or HL60 cells. In other embodiments, the cell or cell lines are iPSCs, including for example: Pgp1, R771, r802, or GR1.1 cells. All these cell lines and/or cells can be modified by methods of the present invention.

Also disclosed herein are systems and kits comprising the disclosed genetically modified cells and configured for performing the disclosed methods. The systems and kits may comprise and/or utilize cryopreserved genetically modified cells, genetic material for a subsequent manipulation event, and devices and/or instructions for using the system and kits.

### Gene Editing

Some aspects of the present disclosure provide methods for manipulating/modifying by gene editing a frozen cell or population of frozen cells after thawing, including contacting a cell or population of cells with the one or more gRNAs or sets of gRNAs, in combination with a nuclease and optionally a donor polynucleotide (e.g., the genetic material), to produce a modified cell or a population of modified cells. The population of modified cells can comprise at least one edit in at least one genomic region of interest. In some instances, contact between the cells and the genetic material, or a genetic manipulation solution containing the genetic material, may occur in a separate container wherein the genetic material, or the genetic manipulation solution, is added prior to the transfer of the cells.

The gRNA, one or more sets of gRNA, can be designed by any of the methods described herein. The at least one edit can result in a knock-out of a gene in the genomic region of interest or a knock-in of a point mutation, an allele, a tag, or an exogenous exon into the genome at the genomic region of interest.

The method can comprise contacting a cell or population of cells that were frozen but not expanded after thawing with the one or more gRNAs or sets of gRNAs and a nuclease, to produce a modified cell or a population of modified cells. The method can further comprise contacting the cell or population of cells with a donor polynucleotide. The contacting can comprise transfecting the one or more gRNAs or sets of gRNAs, the nuclease or a polynucleotide encoding the nuclease, or the combination thereof into the cell or population of cells. In some embodiments, each gRNA in one or more sets of gRNAs is complexed with a Cas protein prior to the transfecting, to produce a Cas-gRNA complex, also referred to herein as CRISPR/Cas complex or CRISPR/Cas system. In some embodiments, the method further comprises transfecting at least one donor polynucleotide into the cell or population of cells. The transfecting can be a nonviral transfection or viral transfection. The nonviral transfection can be electroporation, lipofection, or microinjection. The viral transfection can comprise the use of a viral vector. The viral vector can be a retroviral vector, an adenoviral vector, an adeno associated virus (AAV) vector, an alphavirus vector, a vaccinia virus vector, a herpes simplex virus (HSV) vector, a lentivirus vector, or a retrovirus vector. The viral vector can be a replication-competent viral vector or a replication-incompetent viral vector.

The genomic region of interest can be a gene. The gene can be a gene in a pathway of interest. The method can comprise targeting a plurality of genomic regions of interest. The plurality of genomic regions of interest can comprise a plurality of genes in a pathway of interest. The plurality of genomic regions of interest can comprise a plurality of genes in a plurality of pathways of interest. The pathway of interest can be a metabolic pathway, a signal transduction pathway, or a gene-regulation pathway. The pathway of interest can be a pathway involved in a disease. The disease can be a cancer. The pathway of interest can be a pathway involved in production of a molecule of interest. The molecule of interest can be a molecule with pharmacological activity. The genomic region of interest can be a non-coding region of the genome. The non-coding region can be a regulatory element. The regulatory element can be a cis-regulatory element or a trans-regulatory element. The cis-regulatory element can be a promoter, an enhancer, or a silencer.

In some embodiments, the method includes therapeutically administering the processed cells to a subject having a condition, wherein administration of the cells causes a reduction in at least one indication or symptom of the condition. The condition may include any human or animal disease or symptom of any human or animal disease. For example, the condition may be sickle cell disease (SCD), wherein the genetic manipulation solution is designed to correct a mutation in the beta-globin gene. For instance, the method may include correcting the SCD mutation in a pluripotent/RBC cell producing cell of a human subject, which is then administered (e.g., via injection or implantation) back into the subject. Upon receiving the genetically corrected cells, and the resultant production of healthy RBCs in the subject, one or more conditions of RBCs (e.g., fatigue or pain) is reduced.

In some embodiments, the method comprises contacting a gRNA or set of gRNAs targeting a genomic region of interest with a subset of the population of modified cells. In some embodiments, the method comprises contacting each of a plurality of sets of gRNAs targeting a genomic region of interest with each of a plurality of subsets of the population of modified cells. In one example, a plurality of subsets of the population of modified cells can be placed in each well of a multi-well plate. The multi-well plate can be a 4-well plate, a 6-well plate, a 12-well plate, a 24-well plate, a 48-well plate, a 96-well plate, or a 384-well plate. The subset of the population of modified cells can comprise at least 102, at least 103, at least 104, at least 105, or at least 106 cells. Each well of the multi-well plate can further comprise a set of gRNAs targeting the genomic region of interest. Each set of gRNAs in each well of the multi-well plate can target different genomic regions of interest. The plurality of sets of gRNAs can target at least 5, at least 10, at least 20, at least 50, or at least 100 different genomic regions of interest. In some embodiments, the contacting occurs in each well of the multi-well plate.

In some embodiments, disclosed methods further comprise contacting a population of modified cells or subset of the population of modified cells with a stimulus. The stimulus can be an additional agent. The additional agent can be a therapeutic agent (for example: an antibiotic, biologic, or a small molecule drug) or an agent to induce a disease state in the modified cell.

### Workflow, Batch Processing, and Arraying

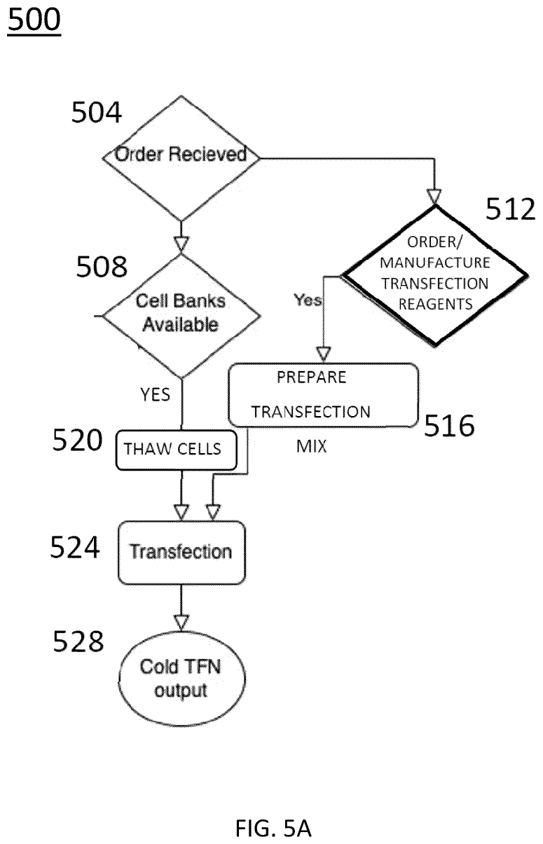

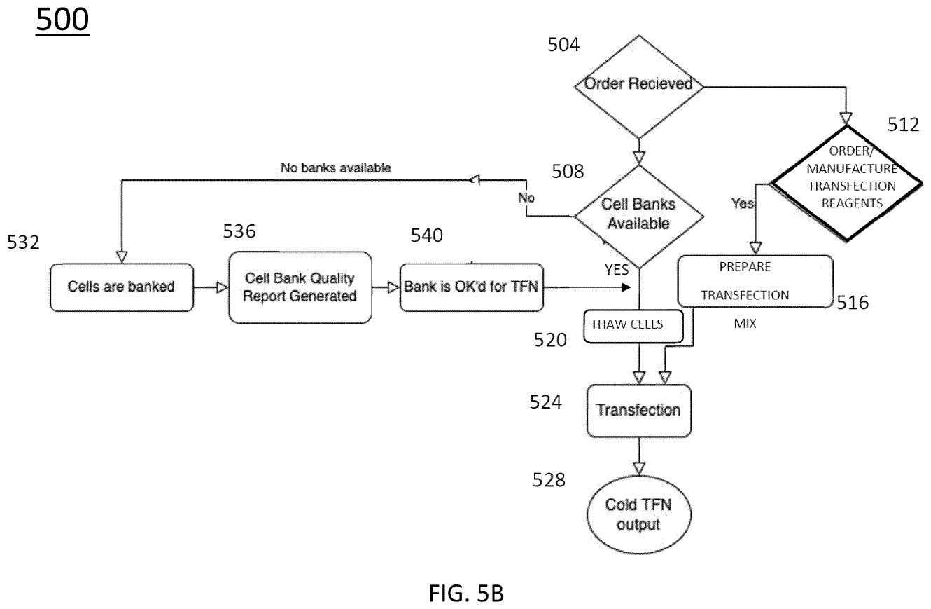

FIG. 5A is a flowchart illustrating a workflow 500 for cold transfections, in accordance with one or more embodiments of the disclosure. The cold transfection may be performed on any type of cell (eukaryotic or prokaryotic) including but not limited to animal cells, plant cells, and fungi cells. For example, the cold transfection may be performed on any type of animal cells including mammalian cells, bird cells, reptilian cells, and amphibian cells. For instance, the cold transfection may be performed on any type of mammalian cells including but not limited to human cells, mouse cells, canine cells, and bovine cells. In particular, the cold transfection may be performed on cells isolated directly from humans or cells expanded from cells isolated from humans (e.g., primary cells) or from human cells lines. The cells may be induced pluripotent stem cells.

In embodiments, the workflow 500 includes a step 504 of receiving an order for performing a transfection (TFN). Once an order for performing a transfection has been received, the workflow 500 proceeds to a step 508 of inquiring if there are cell banks available to perform the transfection. For example, if the order is for the transfection of human cell lines, such as HeLa cells, an inquiry will be made on whether a frozen bank of Hela cells exists that can be used in the transfection. In another example, if the order is for the transfection of patient cells, an inquiry may be made to determine if the patient cells have been received, expanded if necessary, and frozen in one or more aliquots of cells (e.g., a cell bank).

Concurrent to inquiring if the cell bank is available, the workflow 500 further includes a step 512 of ordering and/or manufacturing transfection reagents. Transfection reagents may include one or more of the materials to be transfected (e.g., nucleic acids, proteins, ribonucleoprotein complexes) and the reagents that facilitate transfer (e.g., buffers, lipids). For example, the order may include an endonuclease-based gene editing system, such as an endonuclease-based gene editing system than includes a specific guide RNA (gRNA) sequence for a CRISPR/Cas9 or other nucleic acid-based endonuclease gene editing system transfection, which must be synthesized before the transfection can take place.

Once it is known that the transfection reagents are available, the workflow 500 advances to a step 516 of preparing the transfection mix. For example, for a CRISPR-Cas9 transfection, the transfection mix may include the mixing of at least a gRNA, a Cas9 protein, and a transfection lipid in a transfection buffer. Parallel to the preparation of the transfection mix, one or more aliquots of cells from one of more cell banks are thawed at step 520. Soon after thawing, the transfection mix is applied to the thawed cells in a transfection step 524. After transfection, the cells are incubated further and the effects of the transfection are recorded as a cold transfection output step 528.

If the cell bank is not available at the time of receiving the order, the workflow 500 may further include steps toward creating a cell bank, as shown in FIG. 5B. For example, the workflow 500 may include a step 532 of acquiring cells for banking followed by a step 536 of determining the quality of the banked cells. For example, after the order has been received, cells from a patient sample may be expanded in culture and cryogenically frozen in a series of aliquots. After freezing, one or more aliquots may then be thawed, and the viability of the cells and or transfectability of the cells may be determined and reported in a cell bank quality report. If the results of the cell bank quality report are positive, the workflow 500 may advance to a step 540 of the cell bank being approved for transfection.

The cells may be processed in a batch process. For example, in one or more steps in the method (e.g., workflow 500), more cells are processed (e.g., obtained, thawed, transferred, expanded, or cryopreserved) than are needed for a single use. Batch processing has the advantage of producing extra cell material that can be tested for quality control and for producing cells for multiple uses (e.g., multiple customers). For example, and as shown in FIG. 5B, if there are no cell banks available for a particular cell line, the cells may be expanded as a batch (e.g., expanded to form a cell bank), then cryopreserved in separate tubes. In another example, the cells may be transfected, or otherwise genetically modified, as a batch, and the resultant output of the transfection aliquoted and expanded and/or cryopreserved. In both examples, an aliquot of the batch (e.g., a frozen vial or portion of the transfection) may be used to test the batch (e.g., for cell viability or success of the genetic manipulation). Batch processing may include processing cells in a single large cell container or in a multiplicity of cell containers.

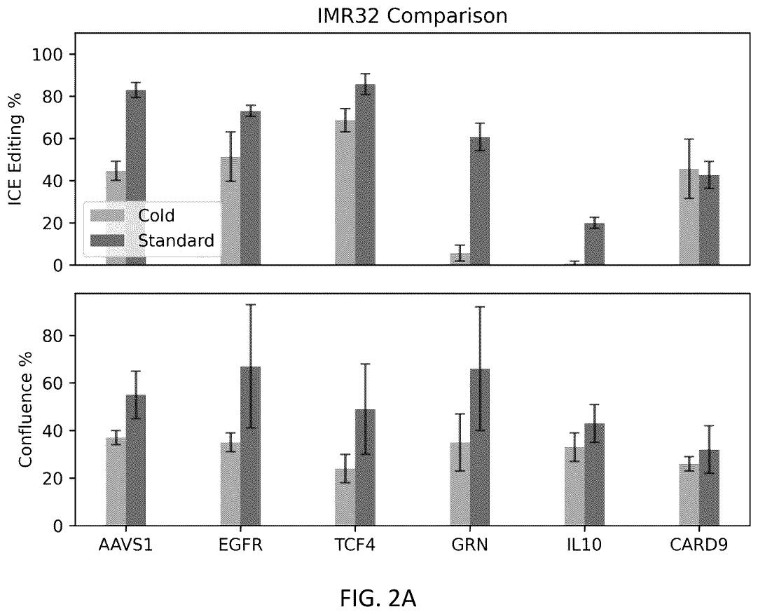

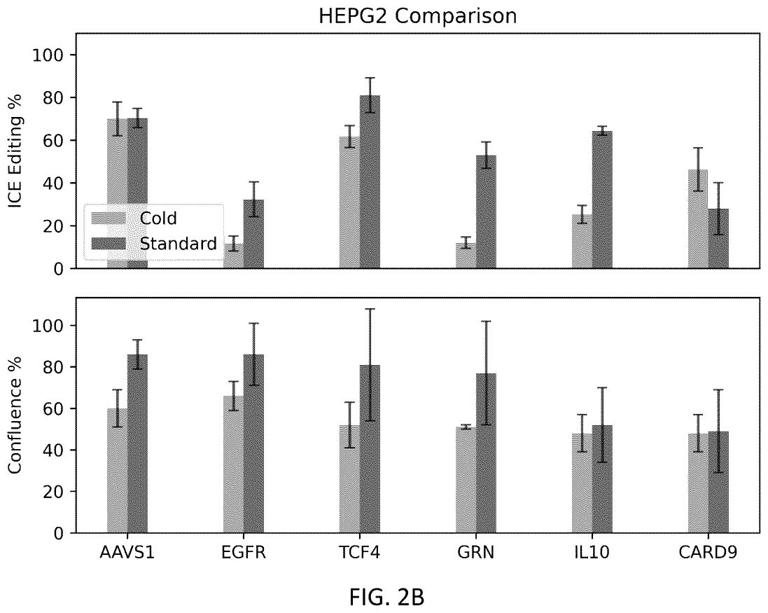

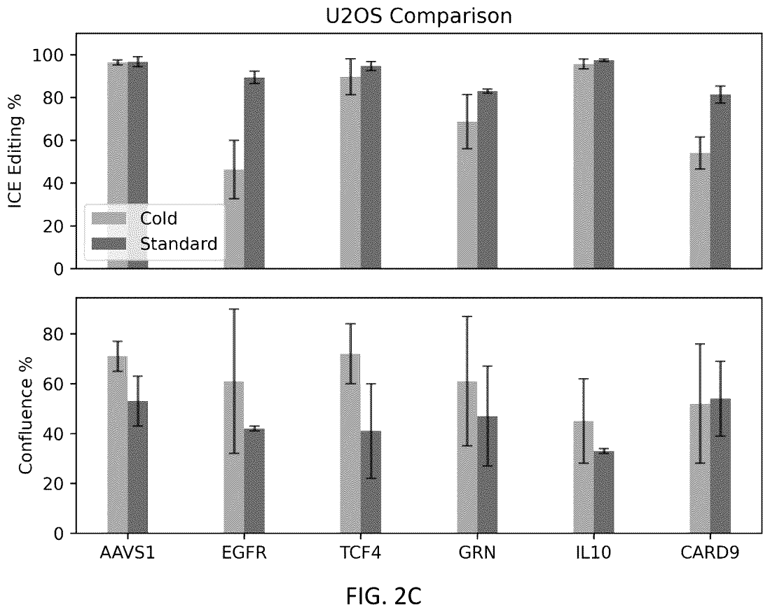

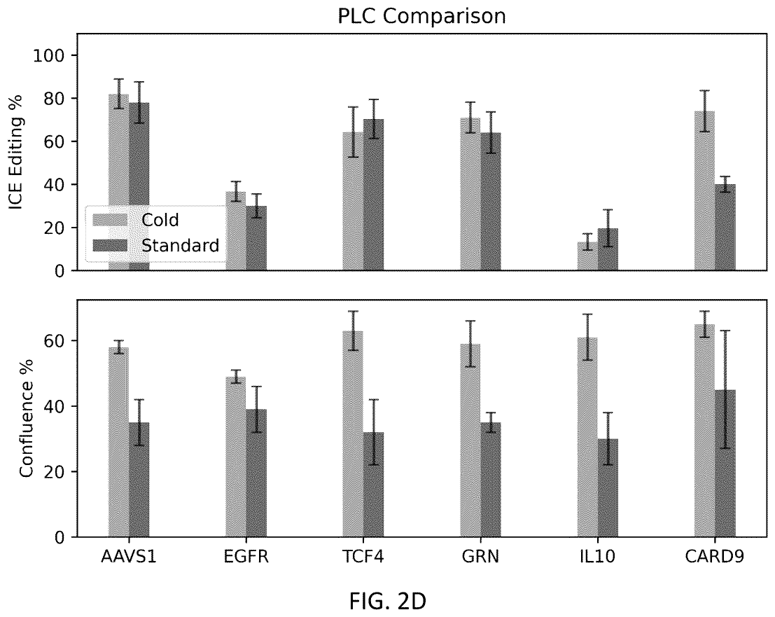

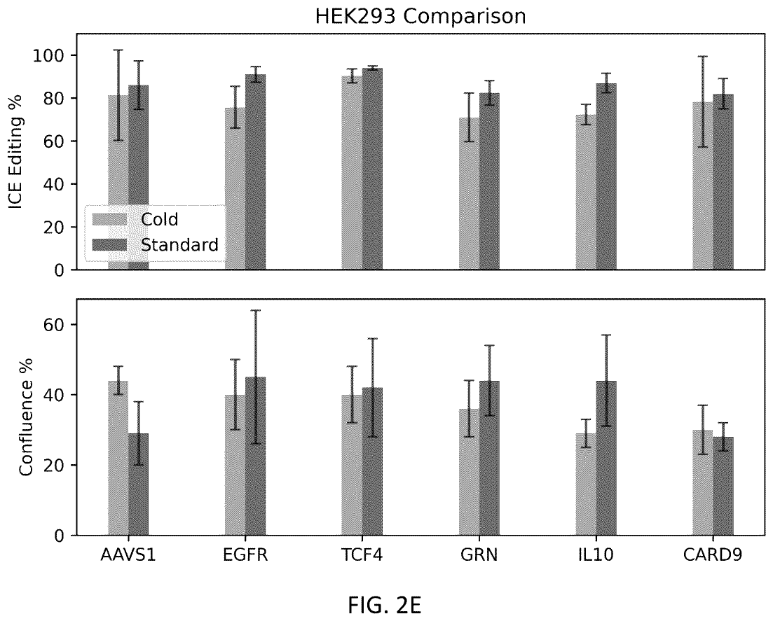

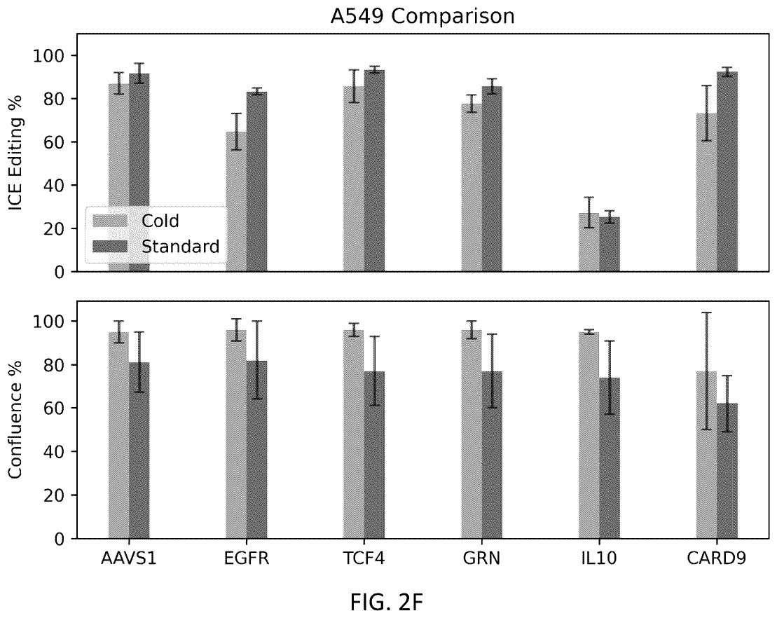

In some embodiments, cell processing is initiated as a batch, which is then divided into individual sub-batches that undergo different cellular conditions. For example, a batch may be initiated in two or more cell containers, wherein the cells in the one or more cell containers are contacted with different genetic material. For example, and as shown in FIGS. 2A-2F, cells may be processed initially as a batch, then aliquoted into separate cell containers to be contacted with cell manipulation solutions for different gene targets. In another example, the same cell types are placed in different cell containers, and the cell editing events comprise editing of different genes in cells from the different cell containers.

In some embodiments, the batch method includes processing of two or more cell containers each having a different cell type, which are then contacted with the same genetic material. For example, primary cells from two individuals suffering from the same genetic disease or mutation may be placed in different cell containers, which are then contacted with cell manipulation solutions designed to cause a genetic mutation that alters or corrects the mutation. In another example, the different cell types or populations are placed in a prearranged position in separate cell container. For instance, the different cell populations may be placed in prearranged wells within a 96-well plate, a 96-well tube-holder, or other arrayed format. When using the same genetic material, the genetic material and/or cell manipulation solution may be generated as a batch, which is then aliquoted for the different cell types.

In some embodiments, the method includes steps for preparing cells that will be used later for batch processing. For example, the method may include a step of expanding cells to form a cell bank. For instance, the cells may be expanded in a growth media to near confluence. In another example, the method may include a step of resuspending the cells in freezing media. Multiple types of freezing media are described herein. In another example, the method may include a step of aliquoting the resuspended cells into a multiplicity of cell containers at a concentration (e.g., 1e6 cells in 100 μL per cell container). In particular, the cell container may have a volume of 1 mL or less. In another example, the method may include a step of freezing the cells in a multiplicity of cell containers and storing the cells. In some cases, each cell container is assigned for a particular gene editing event (e.g., a first gene editing event, a second gene editing event, and so on). In some cases, multiple gene editing events are performed at a given time on a plurality of cells from different cell containers.

Also disclosed herein are methods of preparing frozen previously transfected cells, also referred to herein as Cryogenically Arrayable Stable Transfection-cryo ASap (CASTCas). CASTCas is a method by which the post-transfection plating of cells to allow recovery from the introduction of the genetic material and multiplication of cells described herein are not immediately carried out. Instead, cells are taken from cryogenic storage, thawed, taken into the transfection (genetic introduction) workflow, and then placed back into cryogenic storage without a recovery period, without allowing for multiplication of the cells post-transfection and without allowing sufficient time for substantial editing in the case of transfection (introduction) of a ribonucleoprotein complex for gene editing (Cas and a gRNA) prior to cryogenic storage. In this method, cryopreservation occurs within a short time after transfection and before the cells are allowed to multiply. The cells may be re-frozen less than one hour, less than 30 minutes, less than 20 minutes, less than 15 minutes or less than 10 minutes after the completion of the transfection step. The cells are often frozen before substantial gene editing takes place in the case of introduction of Cas protein and a gRNA. The number of gene edited cells in the cryopreserved cells after transfection may be less than 10% of the cells, less than 5% of the cells, less than 3% of the cells, less than 2% of the cells or less than 1% of the cells. The cryopreserved cells may contain the genetic material transfected into the cell without allowing time for the genetic material transfected into the cells to have an effect on the cell. For example, the ribonucleoprotein complex comprising Cas9 and gRNA may be present within the cryopreserved previously transfected cells.

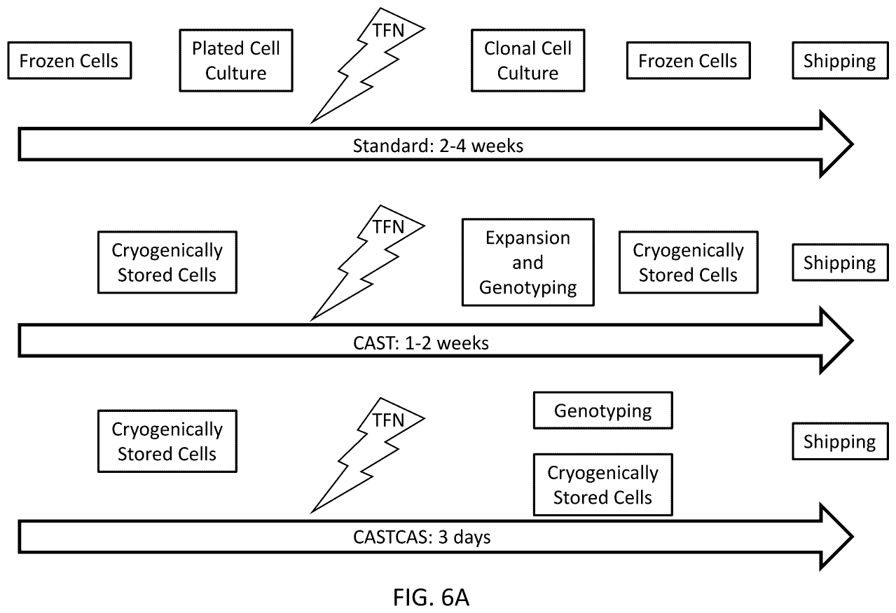

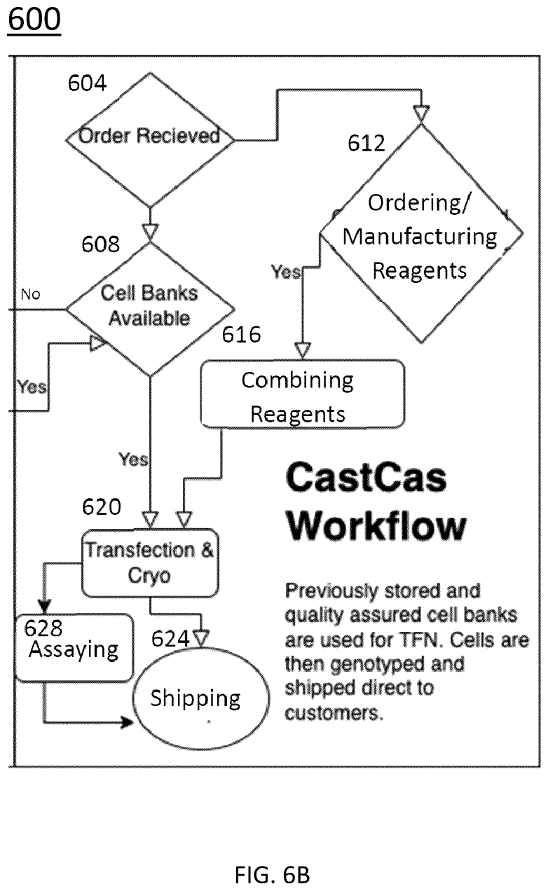

A comparison between the various methods for producing and delivering transfected cells is shown in FIG. 6A. In a standard method of producing and delivering transfected cells (FIG. 6A, Top), cells are plated under cell growth conditions after thawing the cells. Once enough cell growth is obtained, the cells are transfected. Clonal cultures from the transfection are then grown and genotyped. Clones demonstrating successful editing are then packaged and shipped. This method of cell preparation, transfection, clonal isolation, genotyping, and packaging/delivery takes a minimum of 2-4 weeks to perform.

For delivery of CAST cells, cryogenically stored cells are thawed and transfected, without a cell growth step prior to transfection and can be transfected without a centrifugation step. The cells are optionally expanded after transfection (FIG. 6A, Middle). Once the expanded cells are edited, genotyped and optionally successfully edited clones identified, the cells are packaged and shipped. These cells may be cryopreserved prior to shipping. The delivery time of CAST cells is shorter than the standard method, taking only 1-2 weeks from order to shipment.

For delivery of CASTCas cells, cryogenically stored cells are thawed and transfected without a cell growth step prior to transfection and cell can be transfected without a centrifugation step as with the CAST method described above. The transfected cells may be divided into different portions after the transfection. At least one portion of the transfected cells are cryopreserved, packaged, and shipped to a customer or end-user. The cryopreservation after transfection is carried out without providing or allowing an opportunity for the cells to multiply or otherwise recover after transfection and prior to cryopreservation or, in the case of transfection with gene editing materials including Cas protein and gRNA, the cells are not substantially or fully edited prior to cryopreservation. This portion of the cryopreserved cells may be shipped to an end-user or customer. Another portion of the transfected cells may not be shipped, but may be assayed separately for transfection efficiency, editing efficiency, and/or genotyping. This assay data may be sent to the customer or used to evaluate the quality of the transfection or gene editing. After receiving the cryopreserved cells, the customer thaws and plates the cells. The end-user may then evaluate the transfection efficiency, editing efficiency or genotype the cells after the cells are provided an opportunity for editing to occur. If data was generated by the supplier of the cells, the customer may then use the data provided by the supplier as a guide towards determining if the plated cells should have the DNA edit, and how to use the provided cells. The end-user or customer may preform screening, cell growth and cellular cloning assays using techniques commonly used in the fields of molecular and cellular biology and known to those of skill in the art. The obtained cells may be used for any downstream applications of such cells including but not limited to in vitro cell assays or use as a in vivo therapeutic.

Cells generated using CASTCas are delivered considerably more quickly (e.g., approximately three days from placing an order to cell delivery) than the standard method or CAST, as the more lengthy downstream steps which may include editing, genotyping, selection and clonal expansion of a cell occurs after the cells are delivered to the end-user. The method also provides considerable cost cutting over the other methods, as the clonal expansion step can often be performed by the customer at less expense. In CASTCas, cells are taken into cryopreservation post-transfection and are not substantially or completely edited. The ribonucleoprotein (RNP) complex and the complementary cell DNA repair functions (e.g., cell editing functions) are effectively halted at cryopreservation. Once the CASTCas cells are received and thawed, the cell editing functions may resume.

FIG. 6B is a flowchart further illustrating a workflow 600 for the production and delivery of CASTCas cells. The workflow 600 may include one or more steps of workflow 500, and vice-versa. The method 600 includes a step 604 of receiving an order for CASTCas cells. Once an order has been received, the workflow 600 proceeds to a step 608 of inquiring if there are cell banks available to perform the transfection or other introduction of genetic material. If no cell banks are available, the workflow may perform steps 532-540 of workflow 500 as described above. The workflow 600 further includes a step 612 of ordering and/or manufacturing reagents (e.g., guide RNAs and Cas9 protein) required for the transfection or other introduction of genetic material. Once the reagents are available, the workflow proceeds to a step 616 of combining the reagents (e.g., to form RNP complexes) for the transfection.

The workflow 600 then proceeds to a step 620 of transfecting or other introduction of genetic material and then to cryopreserving the cells. The step 620 may include the cryopreservation of one or more portions of cells (e.g., the transfection being divided into two or more portions). At least one portion of the cells is cryopreserved after transfection or other introduction of genetic material and in the case of gene editing before the cells are substantially or completely edited. For example, where the transfection is divided into two or more portions, one or more of the portions may be cryopreserved, halting DNA editing, with one or more portions not cryopreserved and allowed to proceed through the DNA editing. The workflow 600 then proceeds to a step 624 of shipping at least a portion of the cryopreserved cells to an end-user or customer. The shipping of cryopreserved cells may include short-distance or long-distance shipping. For example, short-distance shipping of the cryopreserved cells may include the transport of the cryopreserved cells from one room to another room in the same laboratory or institution or within the same city or local area. Long-distance shipping of cryopreserved cells may include the shipping of cells from a first location to a second location (e.g., the two locations being located in different cities, states, or countries). The cells may be shipped in a suitable container to allow the cells to remain frozen during shipping and to allow for editing to be completed at the intended destination.

Portions of transfected cells to be shipped are cryopreserved immediately after transfection. “cryopreserved immediately after transfection” or “re-frozen” as used herein means less than about 60, 50, 45, 40, 35, 30, 25, 20, 15, 10, 7, 5, 3, 2, 1, or 0.5 minutes after completion of the transfection protocol. This term may also be used to mean that the cells are not stored, plated or allowed an opportunity to recover or multiply or complete gene editing in the case of introduction of gene editing reagents. Once frozen, the cells can be stored indefinitely and can be thawed as described herein to allow the editing event to occur at any given time.

The workflow 600 may optionally include step 628 of assaying the cells (e.g., for transfection efficiency, editing efficiency or genotyping). Assay data may then be sent to the customer (e.g., at the same time or different time than the transfected cells), which informs the customer on the predicted success of obtaining a successful clone from the transfected cells that they have received.

The non-shipped portions of the transfected cells may be cryopreserved, grown, isolated, or assayed many different ways. For example, one or more portions may be cryopreserved and stored as a back-up transfection. In another example, one or more portions may be cryopreserved (e.g., to emulate the cryopreservation of a shipped portion), then thawed and assayed for DNA editing. In another example, the non-shipped cells are not cryopreserved and are cultured for clonal expansion and/or transfection efficiency. In another example, non-shipped cells are not cryopreserved and are cultured and assayed for DNA editing efficiency.

Once the shipped cryopreserved cells are received (e.g., in a different location), the cells can be thawed and plated, allowing the cells to multiply and to resume the DNA editing process initiated by the transfection (e.g., cells transfected with a guide RNA and a Cas protein will be edited once the cells are thawed and plated). The cells may then be selected, clonally expanded and/or genotyped to determine whether the transfected cells have the correct DNA edit. The plated cells may then be cryopreserved for later use or used in assays or for therapeutic applications.

In some embodiments, the transfection includes a selectable or reportable maker that can be used to identify transfected and/or edited cells. For example, the cells may be transfected with a reporter, such as a fluorescent reporter (e.g., GFP, YFP, RFP), that can be selected either manually or by a cell sorting protocol, such as through fluorescent-activated cell sorting (FACS). In another example, the cells may be transfected with a selectable marker, such as an antibiotic resistance gene (e.g., a puromycin resistance gene), that permits the selection of transfected cells via the antibiotic. The reporter or selectable maker may be expressed transiently or stably. The expression of the reporter or selectable marker may be dependent on either the transfection of the gene for the reporter or selectable marker into the cell (e.g., a transfection control), or on the DNA edit itself. For example, a successful DNA edit may include the incorporation of a reporting GFP construct into the genome that is then selected by FACS (e.g., only the edited cells containing GFP are selected).

In some embodiments, cells are taken post-transfection and placed in 0.5 mL tubes before cryopreservation, allowing the tubes to remain in the commonly used Society for Biomolecular Screening (SBS) Format, which is amenable to automation. For example, an Azenta 96-well capper/decapper system can be used to cap and uncap 96 matrix tubes in any combination, and the tubes can be barcoded on the bottom to confirm, monitor, and manage sample identity.

Also disclosed herein are cryopreserved (i.e., frozen) cells comprising a guide RNA and Cas protein (e.g., a CRISPR RNP complex) that have been introduced into the cells by transfection (e.g., electroporation), compositions comprising such cryopreserved cells, and methods of preparing and methods of using such cells. In one embodiment, the cryopreserved cells are pre-edited or substantially pre-edited (e.g., have not been edited by the transfection performed immediately prior). For example, the cells may comprise the components needed for cell editing but the cell editing process has not occurred or is incomplete. In certain embodiments, the components for cell editing include guide RNA and Cas protein introduced into the cell separately or together as a CRISPR RNP complex. A portion of the cryopreserved cells will still contain the cellular genome present prior to the transfection at least until they are thawed. Pre-edited cells may include either cells that have never been edited beforehand, or cells that have been edited previously, but are now undergoing a transfection for another genomic edit. In some embodiments, at least a portion of the cryopreserved cells are not edited by the CRISPR RNP complex.

In some embodiments, the cryopreserved cells comprising guide RNA and Cas protein in a pre-edited or substantially pre-edited state can be prepared from cells that have not undergone the CAST process but rather have been transfected using standard methods known in the art (e.g., the cells have not been thawed and immediately transfected as in CAST but have been undergoing expansion according to conventional methods or are primary cells.)

Accordingly, in some embodiments, a method of preparing cryopreserved cells that comprise a Cas protein and a guide RNA is provided, the method comprising: (i) providing cells; (ii) contacting the cells with the Cas protein and guide RNA and a cell manipulation solution to introduce the Cas protein and guide RNA into the cells; and (iii) freezing the cells before the guide RNA and Cas protein can edit, substantially edit or completely edit the cells. In some embodiments, the cells being provided are cryopreserved and thawed immediately before the contacting step (i.e., undergo the CAST process as provided herein prior to step iii).

### Exemplary Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with an of the preceding or following individually numbered aspects. This is intended to provide support for all such combination of aspects and is not limited to combinations of aspects explicitly provided below.

### EXEMPLARY EMBODIMENTS

1. A method of processing cells comprising:
   (i) obtaining cells, wherein the cells are cryopreserved within a freezing media in a cell container;
   (ii) thawing the cells in the cell container; and
   (iii) contacting the cells with genetic material and a cell manipulation solution to introduce the genetic material into the cells, wherein the thawed cells are transferred to a separate container before step (iii) or after step (iii).
2. The method of claim 1, further comprising removing at least a portion of the freezing media from the cell container after thawing the cells and prior to transferring without disturbing the cells pelleted at the bottom of the cell container, and optionally resuspending the cells before transferring them.
3. A method of processing cells comprising:
   (i) obtaining cells, wherein the cells are cryopreserved within a freezing media in a cell container;
   (ii) thawing the cells in the cell container;
   (iii) removing at least a portion of the freezing media from the cell container after thawing the cells without disturbing the cells pelleted at the bottom of the cell container;
   (iv) contacting the cells with genetic material and a cell manipulation solution to introduce the genetic material into the cells.
4. The method of embodiment 3, further comprising transferring the thawed cells to a separate container after the removing step or after the contacting step.
5. The method of any one of embodiments 1-3, wherein the freezing media is serum-free and is optionally also protein-free.
6. The method of any one of embodiments 2-5, further comprising removing at least 50% of the freezing media from the cell container after thawing and before resuspending the cells or transferring the cells.
7. The method of any one of embodiments 2-6, wherein the volume of the freezing media removed is 90% of an initial volume of the freezing media in the cell container.
8. The method of any one of embodiments 2-7, wherein the removal of the freezing media is by aspiration.
9. The method of any one of embodiments 1-2 or 4-8, wherein the separate container is a well of a multi-well plate or a vial.
10. The method of any one of embodiments 1-2 or 4-9, wherein the contacting step occurs in the separate container.
11. The method of any one of embodiments 1-2 or 4-10, wherein the separate container comprises the cell manipulation solution prior to the cells being transferred.
12. The method of any one of embodiments 1-2 or 4-11, wherein the separate container comprises the genetic material prior to the cells being transferred.
13. The method of any one of embodiments 1-2 or 4-13, wherein the separate container comprises the cell manipulation solution and the genetic material prior to the cells being transferred.
14. The method of any one of the preceding embodiments, wherein the contacting step includes electroporation to introduce the genetic material into the cells.
15. The method of any one of the preceding embodiments, wherein the cells are not washed or centrifuged in or between the thawing step and the contacting step.
16. The method of any one of the preceding embodiments, wherein the cell manipulation solution comprises a transfection solution.
17. The method of any one of the preceding embodiments, wherein genetic material comprises one or more polynucleotides.
18. The method of any one of the preceding embodiments, wherein the genetic material comprises CRISPR ribonucleoprotein complexes.
19. The method of any one of the preceding embodiments, wherein the cell manipulation solution comprises a transfection solution.
20. The method of any one of the preceding embodiments, wherein the cells are processed in one or more batches of cells in a multiplicity of cell containers.
21. The method of embodiment 20, wherein a batch of cells comprises two or more cell containers including cells originating from the same cell population, wherein the cells from each cell container are contacted with different genetic material.
22. The method of embodiment 20, wherein a batch of cells comprises two or more cell containers including cells of at least two differing cell types, wherein the cells in each cell container are contacted with the same genetic material.
23. The method of any one of the preceding embodiments, wherein the cells comprise induced pluripotent stem cells.
24. The method of any one of the preceding embodiments, further comprising therapeutically administering the processed cells to a subject having a condition, wherein administration of the cells causes a reduction in at least one indication of the condition.
25. The method of any one of the preceding embodiments, wherein the genetic material comprises a first gene editing reagent and the cells are further contacted with a second manipulation solution and a second gene editing reagent, wherein the further contacting results in a double knock-out of a selected gene or genes in the cells.
26. The method of any one of the preceding embodiments, wherein the time between the thawing step and the contacting step is less than 60 minutes.
27. The method of any one of the preceding embodiments, wherein the cell container has a volume of 0.25 mL to 0.5 mL.
28. The method of any one of the preceding embodiments, wherein the method is automated after the cells are thawed.
29. The method of any of the preceding embodiments, wherein the cells are frozen prior to step (i) at a concentration of 1e6 cells in 100 μL per cell container.
30. The method of embodiment 29, wherein the cells are frozen at a rate of 1° C./min to −80° C.
31. The method of embodiment 30, wherein after reaching −80° ° C. the cells are placed in the gaseous phase of a liquid nitrogen (LN₂) freezer and acclimated to ambient temperature of approximately −180° C.
32. A method of batch processing of cell editing events in a plurality of cell populations, the method comprising processing a plurality of cell populations according to the method of any one of the preceding embodiments, wherein each cell population is placed in a prearranged position in the separate cell container.
33. The method of embodiment 32, wherein each cell population is the same cell type in different cell containers, and the cell editing events comprise editing of different genes in cells from the different cell containers.
34 The method of embodiment 32, wherein the plurality of cell populations comprises different cell types from cells of different cell containers, and the gene editing events comprise editing of different genes in cells from the different cell containers.
35. The method of embodiment 32, wherein the plurality of cell populations comprises different cell types from cells of different cell containers, and the gene editing events comprise editing of the same gene or genes in each of the cell populations from cells of the different cell containers.
36. The method of any one of the preceding embodiments, further comprising after the contacting step cryopreserving at least a portion of the cells contacted with the genetic material optionally prior to allowing cell multiplication to produce cryopreserved cells.
37. The method of embodiment 36, wherein the cells were contacted in the contacting step with a guide RNA and Cas protein and optionally the cells are cryopreserved before the guide RNA and Cas protein can edit, substantially edit or completely edit the cells.
38. The method of embodiment 36 or 37, wherein a portion of the cells after the contacting step are maintained, optionally not cryopreserved and are genotyped.
39. The method of any one of embodiments 36-38, wherein the cryopreserved cells are shipped frozen to a different location.
40. The method of embodiment 39, wherein the cryopreserved cells are thawed, allowed to multiply and genotyped in the different location.
41. The method of any one of embodiments 1-35, further comprising after the contacting step placing the cells in a tissue culture plate under conditions that the cells multiply.
42. The method of embodiment 41, wherein the cells were contacted in the contacting step with a guide RNA and Cas protein and the guide RNA and Cas protein edit the cells when plated.
43. The method of embodiment 41 or 42, wherein a portion of the cells are genotyped after the contacting step.
44. The method of any one of embodiments 41-43, further comprising cryopreserving the cells after multiplication.
45. The method of any one of embodiments 41-44, further comprising shipping the cells to a different location.
46. A method of preparing cells for batch processing of cell editing transfections, the method comprising:
   (i) expanding cells to form a cell bank;
   (ii) resuspending the cells in a freezing media, optionally a serum-free and/or protein-free media;
   (iii) aliquoting the cells from step (ii) into a multiplicity of cell containers at a concentration of 1e5 to 1e7 cells in 100 μL per cell container, wherein each cell container has a volume of 1 mL or less;
   (iv) freezing the cells in the multiplicity of cell containers to prepare cryopreserved cells; and
   (v) storing the cryopreserved cells for cell editing transfections.
47. The method of embodiment 46, wherein each cell container is assigned for a particular gene editing event.
48. The method of embodiment 47, wherein a cell container is assigned for a second gene editing event.
49. The method of any one of embodiments 46-48, wherein multiple gene editing events are performed at a given time on a plurality of cells from different cell containers.
50. The method of any one of embodiments 46-49, wherein each container has a volume of 0.25 mL to 0.5 mL.
51. The method of any one of embodiments 46-50, further comprising processing at least one container of cells using the method of any one of embodiments 1-45.
52. The method of embodiment 51, wherein the processing of the cells is used to calculate an efficiency of processing for the batch of cells.
53. A method of high-throughput cell editing comprising:
   (i) providing a plurality of arrayed cryopreserved cell populations;
   (ii) thawing the arrayed cell populations; and
   (iii) contacting the arrayed cell populations with genetic material and a cell manipulation solution to introduce the genetic material into the cells of the cell populations, wherein the contacting occurs within one hour of thawing.
54. The method of embodiment 53, wherein the cell populations are provided in cell containers at a concentration of 1e5 to 1e7 cells in 100 μL per cell container.
55. The method of embodiment 53, wherein each cell population is the same cell type.
56. The method of embodiment 53, wherein the plurality of cell populations comprises various cell types.
57. The method of any one of embodiments 53-56, wherein each arrayed cell population is assigned for a particular gene editing event.
58. The method of embodiment 57, wherein each arrayed cell population is assigned for a second gene editing event.
59. The method of any one of embodiments 53-58, wherein gene editing is performed at a given time on the plurality of arrayed cell populations.
60. The method of any one of embodiments 53-59, wherein the cryopreserved cells were frozen in a serum-free freezing media, and wherein the media is optionally also protein-free.
61. A composition comprising cryopreserved cells, wherein the cells comprise a guide RNA and a Cas protein introduced into the cells by transfection.
62. The composition of embodiment 61, wherein the cells have not been completely edited.
63. The composition of embodiment 61 or 62, wherein a majority of the cells contain the cellular genome present prior to addition of the guide RNA and Cas protein.
64. The composition of any one of embodiments 61-63, wherein the cells are made by any one of the methods of embodiments 1-52.
65. The composition of any one of embodiments 61-64, further comprising a serum-free freezing media, wherein the media is optionally also protein-free.
66. A method of using the composition of any one of embodiments 61-65, further comprising thawing the cells and allowing the cells to be edited.
67. The method of embodiment 66, further comprising selecting edited cells.
68. A method of preparing cryopreserved cells that comprise a Cas protein and a guide RNA, the method comprising:
   (i) providing cells;
   (ii) contacting the cells with the Cas protein and guide RNA and a cell manipulation solution to introduce the Cas protein and guide RNA into the cells; and
   (iii) freezing the cells before the guide RNA and Cas protein can edit, substantially edit or completely edit the cells.
69. The method of embodiment 68, wherein the cells being provided are cryopreserved and thawed immediately before the contacting step (ii).
70. The method of embodiment 68 or 69, wherein the Cas protein and guide RNA are introduced by transfection.

### EXAMPLES

### Example 1

### Cryogenically Arrayable Stable Transfection (CAST) Process Compared to Standard Transfection Process

To determine if cryogenically prepared cells could be transfected without the time-consuming process of cell expansion post thawing, cells were subjected to an accelerated transfection process as described in this Example and compared to cells transfected according to a standard process. In particular, the accelerated transfection process was assessed for use in CRISPR gene editing.

### Cryogenic Cell Prep for Standard Transfection

Cell vials with 1E6 cells (IMR32, HEPG2, U2OS, PLC, and HEK293) were removed from storage in liquid nitrogen, thawed via a Thawstar thawing device (Biolife Solutions) and plated into T75's to be used in future banks (these banks generally consisting of 10-15 matrix tubes of 1E6 cells each) (this generally involved 1-2 passages).

The cryopreservation process involved taking the bankable flasks of cells and performing multiple washes with Dulbecco's phosphate-buffered saline (dPBS), and using TrypLE solution (Thermo Fisher Scientific, Inc.), to remove the cells from the flask. After the cells were disassociated from the flask, an equivalent volume of cell media was added to quench the TrypLE enzymes. The cell solution was counted for an estimated cell number, and all the available volume of cells/mL was taken and spun down in a 15 ml conical centrifuge tube. The cells formed a pellet at the bottom of the centrifuge tube and the remaining media/TrypLE solution was aspirated and tossed. The cells were then resuspended with the desired amount of Thermo Fisher Recovery Cell Culture Freezing media and aliquoted at a concentration of 1E6 cells/1 mL into 2 mL cryovials. These vials were frozen at an approximate rate of 1° C./min to −80° C., after which they were taken and placed above gaseous liquid nitrogen (LN₂) where they acclimated to the ambient temperature of approximately −180° C.

To begin the preparation for the standard transfection (StdTFN) process, cell banks were removed from storage and the thawing and plating steps were begun. Cell vials were removed from the LN₂ tanks and placed on dry ice to await thawing. The thawing step was done using the BIOLIFE Solutions ThawStar (Biolife Solutions Inc., Bothell, WA). To dilute the percentage of cryomedia present in the ultimate flask, the cells were gently mixed and aliquoted into 10 mL of 10% FBS DMEM:F12+Glutamax Cell media (Gibco™ 10567022). The cells were then spun down in a centrifuge tube. The remaining media was aspirated from the cell pellet. Fresh cell media was used to resuspend and transfer the cells into a T25 cell flask. After 2-5 days, cells were dissociated from the T25 using TrypLE and plated into a larger T75 flask until they were ˜70-80% confluent. This process of growth generally took around 1-2 weeks and generally involved 1-2 hours or more of technician touch/handling time.

Once the cells were confluent, they were dissociated from the flask (following the procedure described above). 2E6 cells are pelleted via centrifugation per 12 samples (1 row in a 96 well plate). The cells were then resuspended in 80 μL of SF Lonza buffer (V4XC-2032) and placed into the first column of an empty 96 well plate. This was referred to as the ‘cell source’ plate and was placed into a Hamilton automated liquid handler (Hamilton Company, Reno NV) as illustrated for example in FIG. 1.

For the transfection, CRISPR reagents were generated to edit the AAVS1 gene in the IMR32, HEPG2, U2OS, PLC, and HEK293 cells. First, a ribonucleoprotein (RNP) complex was made using a guide RNA targeting the gene and the spCas9 nuclease (Aldevron). To generate the RNP complex SF Lonza buffer (V4XC-2032) was mixed with spCas9. From here 34.5 μL of the mixture was aliquoted to all wells of an empty 96 well Hamilton full skirt PCR plate (Hamilton Company, Reno NV). After this sgRNA was added to generate the RNP complex. This plate was referred to as the ‘RNP Plate’ as illustrated in FIG. 1.

The automated Hamilton method was used to aliquot cells from the cell source plate across the mixing plate (an empty 96 well Hamilton full skirt plate). Each row of wells of the cell source plate was broadcasted to corresponding wells of the mixing plate (1 row). The RNP mixture of the RNP Plate is picked up and mixed in the mixing plate, then dispensed into a 96-well Shuttle® from Lonza (Lonza AG, Basel, Switzerland). The shuttle is moved to an Amaxa® Nucleofector® system on the automatic liquid handler (Hamilton deck) and nucleofected according to the manufacturer's protocol. 10% FBS DMEM:F12+Glutamax Cell media (Gibco™ 10567022) from a media trough was added and the transfected cells were broadcasted to three empty 96 well plates. The three 96 well tissue culture plates were taken into a 37° C. incubator with 5% CO₂. They were stored and genotyped on the third day to allow the editing time to take place. Editing efficiency was measured by Sanger and Inference of CRISPR Edits (ICE).

### Cryogenic Cell Prep for CAST

Cell vials with 1E6 cells (IMR32, HEPG2, U2OS, PLC, and HEK293) were removed from storage in liquid nitrogen, thawed as above and plated into T75's to be used in future banks (these banks generally consisting of 10-15 matrix tubes of 1E6 cells each) (this generally involved 1-2 passages).

The bankable flasks of cells were washed multiple times with dPBS, and TrypLE solution was used to remove the cells from the flask. After the cells were disassociated from the flask, an equivalent volume of 10% FBS DMEM:F12+Glutamax Cell media (Gibco™ 10567022) was added to quench the TrypLE enzymes. The cell solution was counted for an estimated cell number, and the total volume of cells/mL was taken and spun down in a 15 ml conical centrifuge tube. The cells formed a pellet at the bottom of the centrifuge tube and the remaining media/TrypLE solution was aspirated and tossed. The cells were then resuspended with the cryomedium for a concentration of 1e6 cells/100 μL and moved into 96 well matrix tubes each having a volume of 0.5 mL. The vials were frozen at an approximate rate of 1° C./min to −80° C., after which they were placed above gaseous liquid nitrogen (LN₂) where they acclimated to the ambient temperature of approximately −180° C.

CRISPR RNP complexes were generated as described above for the StdTFN. A Hamilton automated liquid handler was prepared as illustrated in FIG. 1 and as described above, except the cell source plate was a 96 well matrix plate with up to 8 tubes (1 column) instead of a 96 well full skirt plate. The cells were transfected as described above. Cell editing efficiency was measured as described above.

### Results

The editing results were comparable between the processes. See FIGS. 2A-2F.

### Example 2

### Optimal Volume of Freezing Media

This example was conducted to assess the impact of the freezing media volume on transfection efficiency in frozen cells subjected to the CAST system described herein.

HEK293 cells were banked at a desired concentration of 1e6 cells in 100 μL per matrix tube as described in Example 1. To bank the cells, they were grown until 70-80% confluent before being washed and trypsinized to dissociate from the cell flask. They were then counted and spun at 200 g for 5 minutes, before being resuspended in freezing media (FrM) and aliquoted 1E6 cells in 100 μL per tube (a total of 1 tube).

The CRISPR RNP complexes were generated well by well as described above. To each well spCas9 and sgRNA were added, then another 32.8 μL of SF Buffer (V4XC-2032) with a given percentage of FrM, for a total of 36 μL reaction volume, and the cells were transfected in the Hamilton automated liquid handler as described above, along with the banked cells. The volumes 5 of 12 samples are shown in Table 1.

TABLE 1

| | | 1 | 2 | 3 | 4 | 5 | 6 | 7 | 8 | 9 | 10 | 11 | 12 |
|---|---|---|---|---|---|---|---|---|---|---|---|---|---|
| % FrM in Cell | A | 0 | 5 | 10 | 15 | 25 | 50 | 0 | 5 | 10 | 15 | 25 | 50 |
| Source | | | | | | | | | | | | | |
| Volume SF to add: | | 32.8 | 31.2 | 29.5 | 27.9 | 24.6 | 16.4 | 32.8 | 31.2 | 29.5 | 27.9 | 24.6 | 16.4 |
| Volume FrM to add: | | 0 | 1.6 | 3.3 | 4.9 | 8.2 | 16.4 | 0 | 1.6 | 3.3 | 4.9 | 8.2 | 16.4 |
| Final FrM conc: | | 0.17 | 0.2 | 0.24 | 0.28 | 0.36 | 0.55 | 0.17 | 0.2 | 0.24 | 0.28 | 0.36 | 0.55 |
| Total Volume | | 32.8 | 32.8 | 32.8 | 32.8 | 32.8 | 32.8 | 32.8 | 32.8 | 32.8 | 32.8 | 32.8 | 32.8 |
| (FrM + SF buffer) | | | | | | | | | | | | | |
| [w/o spCas9 or | | | | | | | | | | | | | |
| sgRNA] | | | | | | | | | | | | | |

### Results & Discussion:

Editing efficiency was measured by ICE.

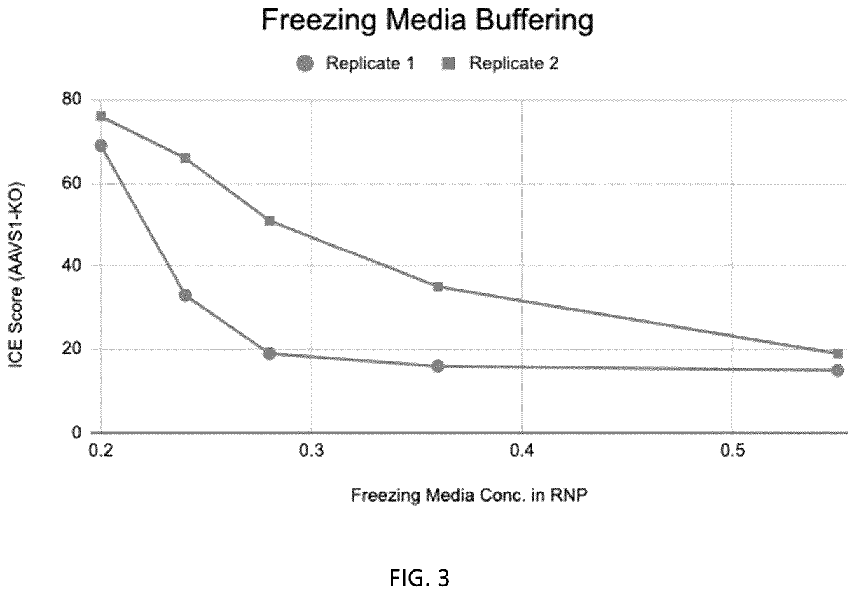

The results showed that editing efficiency of CRISPR RNP complexes decreased rapidly between concentration of 0.2 and 0.3. See FIG. 3.

### Example 3

### CAST System to Generate Double Knock-Out Cells

To test the CAST system in making double knock-out cell edits, HEK293 cells were seeded into a 96 well plate as follows: 50,000 cells were plated into the first well and split in half in every following well (i.e., 25,000 in the next well, 12,500 in the following well, etc.). The cell number was also estimated later using the confluence of the well. These were left in a 5% CO₂-37° C. incubator overnight before being frozen using a Hamilton automated liquid handler for 96 well plates according to set specifications. One column of the cryogenically frozen wells was taken and thawed for use in the CAST system.

To generate the RNP complex, 3.5 mL of SF (V4XC-2032) buffer was mixed with spCas9. Then 34.5 μL of the mixture was aliquoted to all wells of an empty 96 well Hamilton full skirt PCR plate (Hamilton Company, Reno NV). Afterward, AAVS1 sgRNA was added to generate the RNP complex.

The CAST system applied in this Example differed slightly from that described above as it involved an aspiration step after thawing and before placing into a source plate. After thawing as above, the freezing media was aspirated, being careful not to disturb the cell pellet. The cells were then resuspended in 25 μL of SF Buffer (V4XC-2032) and transferred to well of a source plate in a Hamilton automated liquid handler as described above. Because there was an increased proportion of Lonza buffer than for a usual transfection, the media was changed after day 1 with fresh 10% FBS DMEM:F12+Glutamax Cell media (Gibco™ 10567022). To estimate the actual number of cells leftover, some of the remaining tubes were thawed and counted. Their measured confluence was used in a standard curve to estimate the number of cells for a given confluence (this was done to account for losses in the cryopreservation method, which is a known effect, where the Hamilton may only successfully take 70-90% of the available cells).

### Results:

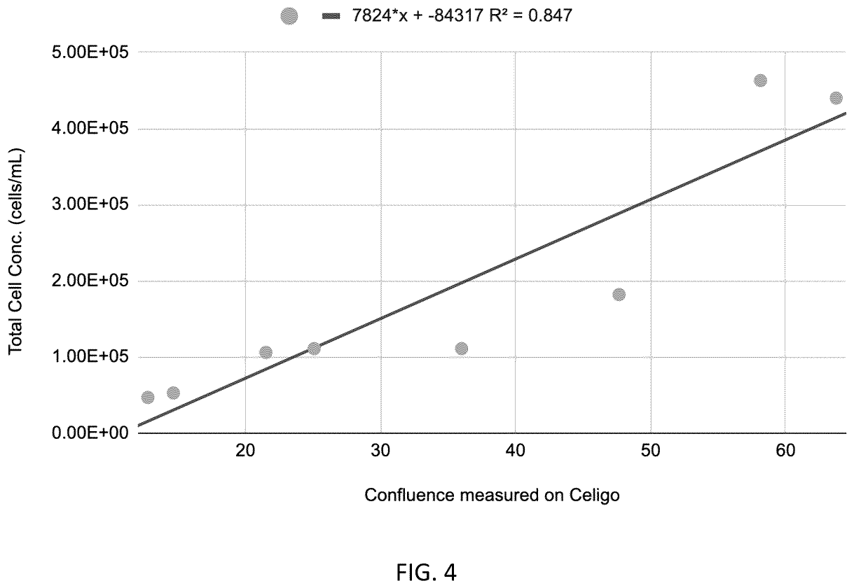

Editing efficiency in the cells was measured by Sanger and ICE. Results are shown in FIG. 4, which confirms the ability of the CAST system to successfully transfect extremely low concentrations of cryopreserved cells, demonstrating its potential as an effective tool for use in creating double knock-outs, as well as in cases where a high concentration of cells at cryopreservation is not feasible.

### Example 4

SNV & iPSC Editing

To determine if the CAST system can be used to create a single nucleotide variation (SNV) using a template donor sequence in iPSCs, four iPSC cell lines were prepared for the CAST system protocol, r771 (Reprocell-RCRP004N), r802 (Reprocell-RCRP005N), GR1.1 (NHLBI-NN0005200), PGP1-SVI (Dr. George Church, Harvard University). The cells were grown in T75 flasks in Thermo Fisher Stemflex (A3349401) media with CEPT cocktail (Chroman 1, Emricasan, Polyamine solution, and Trans-ISRIB) and iMatrix as a substrate, as described in the art.

After 2-5 days, cells were then frozen using the standard cryopreservation protocol described above with the following changes: Accutase (ICT, CAT #AT104-500) was used instead of TrypLE to dissociate the cells. The Knock-out Serum (KSR) (ThermoFisher, CAT #10828028) was used instead of the standard Freezing media. Cells were washed with dPBS and left in 37° C. for 15 minutes with 7 mL of Accutase to dissociate. Once dissociated, an equivalent volume (7 mL) of Stemflex was added to quench the reaction. Cells were counted and the total volume was moved to 15 ml conical tube and spun for 5 minutes at 200 g. The remaining media was aspirated carefully, to not disturb the pelleted cells, and then the remaining cells were resuspended to a concentration of 1E6 cells/100 μL using the KSR freezing media. 100 μL was of the suspension was aliquoted to 20 of the 96w Matrix tubes, which were then capped and frozen at an approximate rate of 1° C./min to −80 C. After which they were moved to the gaseous phase above liquid nitrogen.

The cells were thawed in a liquid at room temperature for 2-4 minutes until thawed and the arrayed vials were then placed into the Hamilton deck. After which the standard CAST method was used (including the initial freezing media aspiration).

To generate the RNP complex, 3.5 mL of Lonza P3 buffer (CAT #V4XP-3024) was mixed with spCas9. Then 34.5 μL of the mixture was aliquoted to all wells of an empty 96 well Hamilton full skirt PCR plate (Hamilton Company, Reno NV). After this the desired sgRNA was added to generate the RNP complex, and the desired single-stranded oligodeoxynucleotide (ssODN) was added to the RNP plate (as illustrated in FIG. 1). There was a total of 6 gene targets arrayed onto the RNP plate, each of these targets had a specific sgRNA and corresponding ssODN that needed to be added. These genes were: AAVS1, EGFR, TCF4, GRN, IL10, and CARD9. Cells were placed into a 32° C. incubator for 24 hours post transfection (known as a ‘cold shock’). Gene editing was measured as above.

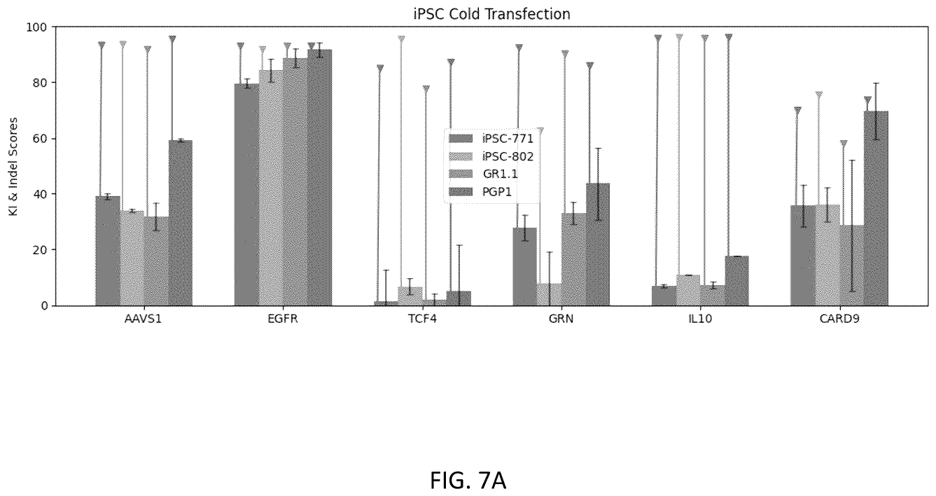

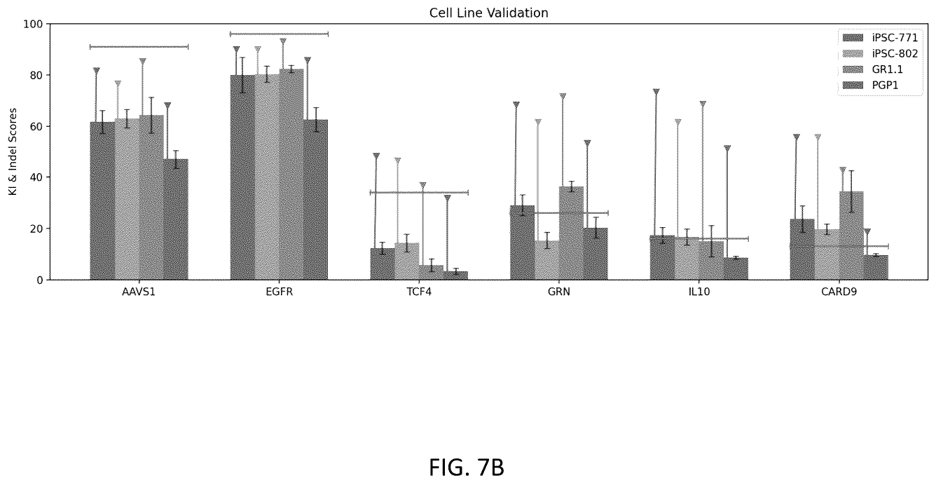

As shown in FIGS. 7A-B, the CAST system was able to successfully modify the AAVS1, EGFR, TCF4, GRN, IL10, and CARD9 gene in the iPS cells.

### Example 5

Rapid Freezing of Cells from CAST System

Cell vials containing several different cell lines were received and cells were grown into large numbers in flasks. Cells were then dissociated from the flasks, resuspended with cryomedium, and moved into 96 well matrix 0.5 mL tubes at a concentration of 1e6 cells in 100 μL per matrix tube.

The matrix tubes were frozen at an approximate rate of 1° C./min to −80° C., after which they were taken and placed above gaseous liquid nitrogen (LN₂) where they were allowed to acclimate to the ambient temperature of approximately −180° C.

To generate the RNP complex, 3.5 mL of SF buffer (Lonza) was mixed with 50 μL of 60 nM Cas9. 34.5 μL of the mixture was aliquoted to all wells of an empty 96 well Hamilton full skirt PCR plate. 1.5 μL of 60 nM of a representative sgRNA was then added to generate the RNP complex. This plate was referred to as the ‘RNP Plate’.

The tube sources containing frozen cells were re-arrayed into the first column of a 96w matrix plate and thawed under 37 C.° forced air.

Cells were aliquoted from the cell source and dispensed across the mixing plate (the mixing plate was an empty 96 well Hamilton fullskirt). Each well of the cell source was broadcasted to 12 wells of the mixing plate (i.e., one row). The RNP mixture of the RNP Plate was picked up and mixed in the mixing plate, then dispensed into the Nucleocuvette™ plate. The Nucleocuvette™ plate was moved to the Amaxa nucleofection system (e.g., the Amaxa™ 96-well Shuttle™) on the Hamilton deck and nucleofected per manufacturer's instructions. 70 μL of media from a media trough was added to 30 μL of cells in LZ buffer and the nucleofected cells were broadcasted to 3 plates. One of the plates was a 96 well tissue culture plate.

To proceed with the CAST process, the 96 well tissue culture plate was used to grow the cells and allow editing time to take place. The plate was taken into a 37 C.° incubator with 5% CO₂, incubated for 3 days, and genotyped on the third day. Editing efficiency was measured by ICE. The editing efficiency of this plate can be considered representative of the two cryo-destination plates.

To demonstrate the CASTCas process (i.e., freezing cells after transfection but before cell editing takes place), the other two plates were 96 well matrix tube plates for cryopreservation. Cells from the Lonza plate were taken and 30 μL of the nucleofected cell source was added to 100 μL of CS10 cryomedia. Both plates were frozen using the same standard process (e.g., frozen at 1° C./min to −80° C. and stored in the gas phase above LN₂). One plate was considered a ‘retain’, meant to be used only when necessary as a backup. The other destination was plated after >1 day in the LN₂ (this is done to examine the editing efficiency and health of the cells, cells stored in the LN₂ can be stored there indefinitely with no significant changes in viability after thaw). Cells were thawed with 37 C.° forced air—the same process used to thaw the cell banks for transfection in CAST. The 130 μL volume was taken into a 24-well plate with at least 2 mL of cell media. This was done to dilute the DMSO to <1% of the total volume while avoiding any centrifugation steps. Cells were grown using the same requirements as the ‘plated’ destination: 37 C.° incubator with 5% CO₂, incubated for 3 days, and genotyped on the third day. Editing efficiency was measured by ICE.

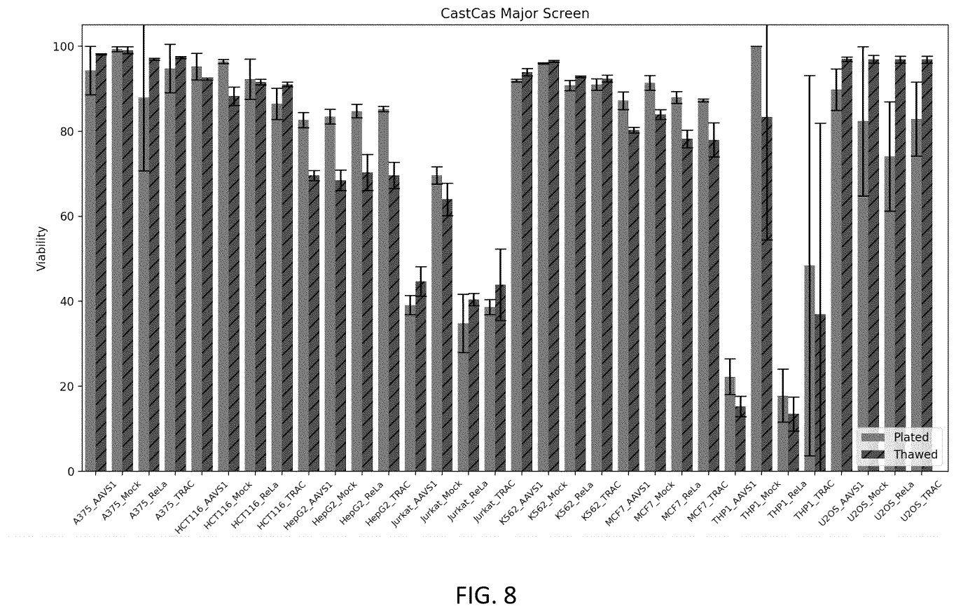

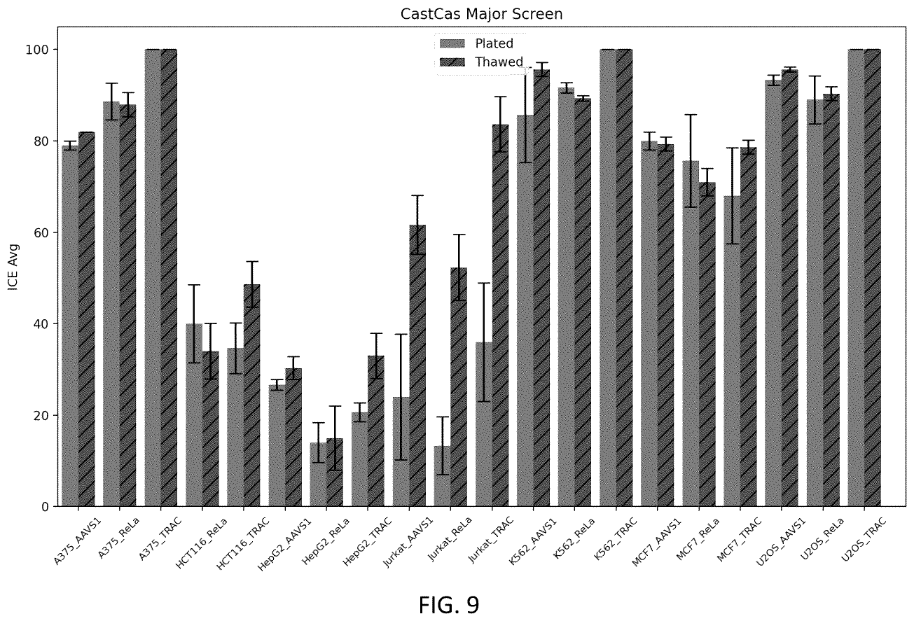

In FIG. 8 and FIG. 9 the immediately ‘plated’ category demonstrates the CAST process, while the ‘thaw’ demonstrates the CASTCas process. As shown in FIG. 8, the viability of the cells undergoing CAST and CASTCas is similar. As shown in FIG. 9, the editing results were comparable between the CAST and CASTCas processes. The two categories resulted in very similar editing efficiencies. The standard deviations (n=3) generally had thawed (CASTCas) performing slightly better than the plated (CAST) outcomes.

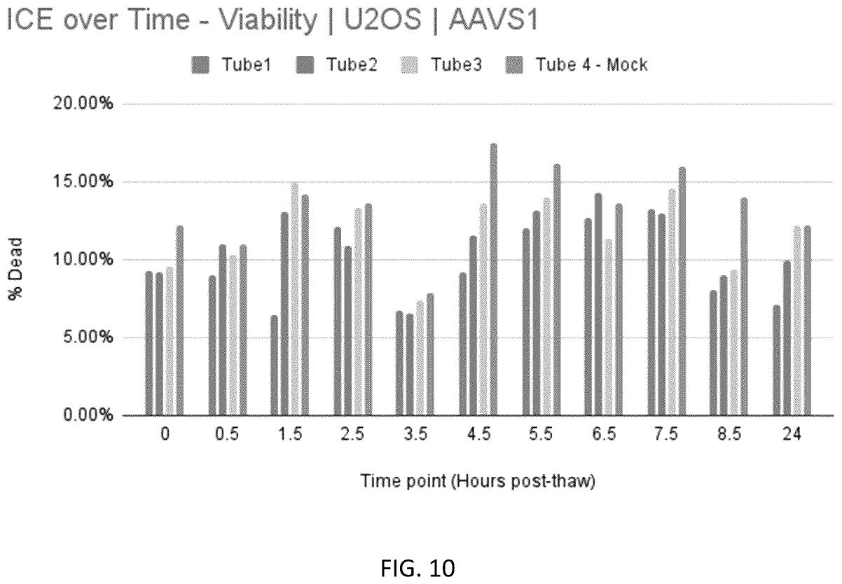

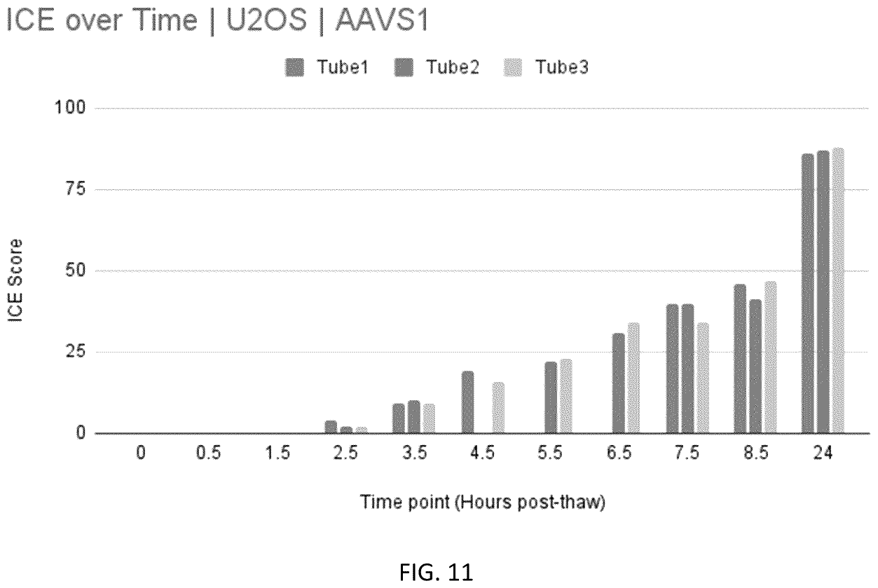

FIG. 10 and FIG. 11 show the percent of cells from CASTCas protocol that were viable (FIG. 10) and edited (FIG. 11). The results show that no editing events were detectable until about 2.5 hours after thawing the CASTCas cells.

### Example 6

### Testing for Freezing Media Effects on Transfection in CAST

The following experiments were conducted to determine whether a single cryo media component specifically affects transfection of cells in the CAST process described herein.

HEK293-RFP cells were grown in standard conditions: 37 C, 5% CO₂, DMEM+10% FBS. The day of transfection, cells were dissociated with TryplExpress (Invitrogen), pelleted by centrifugation, then resuspended in Lonza buffer SF at a concentration of 1E7 cells/ml. 5 ul of cell suspension was added to pre-assembled reaction buffer, and the cells were transfected using Lonza SF-CM-137 protocol. After 48 hours DNA was isolated and PCR for the AAVS1 locus was performed using Primers: 5′-TCCTCTCTGGCTCCATCGTA-3′ (SEQ ID NO: 1) and 5′-CCGTTCTCCTGTGGATTCGG-3′ (SEQ ID NO: 2). PCR products were purified and subject to sanger sequencing using primer 5′-GCAAACCTTAGAGGTTCTGG-3′ (SEQ ID NO: 3). Sequencing results were evaluated using Synthego's ICE tool (Synthego Corp).

Each transfection reaction contained Ribonuclear Protein complex (RNP) consisting of 20 pMol recombinant Cas9 (Aldevron), complexed with 100 pMol AAVS1 sgRNA sequence ACCCCACAGUGGGGCCACUA (SEQ ID NO: 4) in 15.5 ul Lonza SF buffer. Mock transfection reactions were performed with Cas9 protein alone in Lonza SF buffer. Each test additive was then added to the 15.5 ul reaction, and total volume was increased to 30 ul by addition of Lonza SF buffer. Knockout Serum Replacement (KSR) (Thermo Scientific), DMEM:F12 (Invitrogen), Dimethylsulfoxide (DMSO), Freezing media (10% DMSO, 50% KSR, 40% DMEM:F12) were tested at increasing percent of total transfection volume.

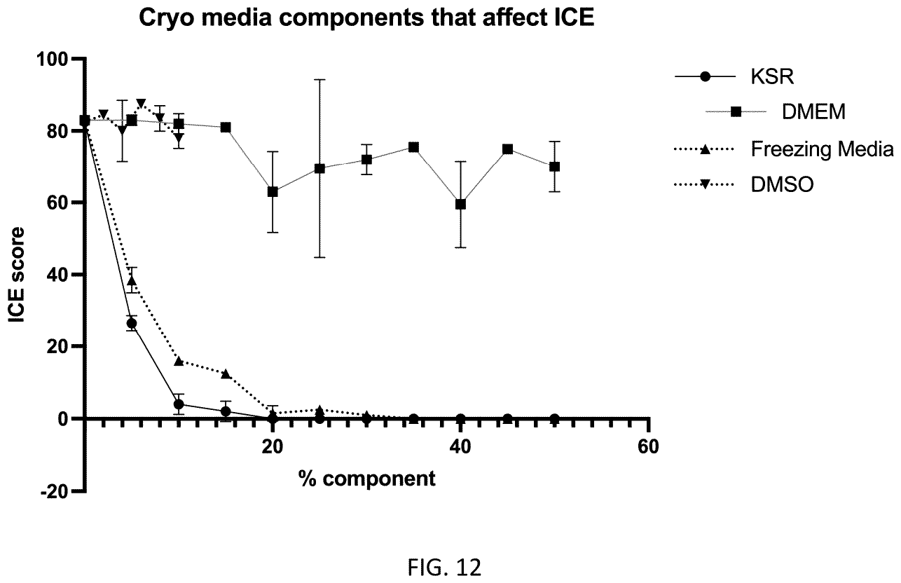

Results are shown in Table 2 and FIG. 12.

TABLE 2

| Condition | 1 | 2 | 3 | 4 | 5 | 6 | 7 | 8 | 9 | 10 |
|---|---|---|---|---|---|---|---|---|---|---|
| Control | AAVS1 | AAVS1 | AAVS1 | AAVS1 | AAVS1 | mock | mock | mock | mock | mock |
| KSR | 5% | 10% | 15% | 20% | 25% | 30% | 35% | 40% | 45% | 50% |
| KSR | 5% | 10% | 15% | 20% | 25% | 30% | 35% | 40% | 45% | 50% |
| DMEM:F12 | 5% | 10% | 15% | 20% | 25% | 30% | 35% | 40% | 45% | 50% |
| DMEM:F12 | 5% | 10% | 15% | 20% | 25% | 30% | 35% | 40% | 45% | 50% |
| Freeze | 5% | 10% | 15% | 20% | 25% | 30% | 35% | 40% | 45% | 50% |
| Media | | | | | | | | | | |
| Freeze | 5% | 10% | 15% | 20% | 25% | 30% | 35% | 40% | 45% | 50% |
| Media | | | | | | | | | | |
| DMSO | 2% | 4% | 6% | 8% | 10% | 2% | 4% | 6% | 8% | 10% |

The results indicated that transfection was largely insensitive to DMSO or DMEM:F12 (2 of 3 cryo media components), but was sensitive to low levels of KSR.

### Example 7

### Testing for Freezing Media Component Effects on Transfection in CAST

As the results above showed that Knockout Serum Replacement (KSR) in the tested media blocks CAST transfection even at low concentrations. To further explore impact of media components on CAST transfection, the following experiments were conducted to determine whether Serum free commercially available cryo media support CAST transfection, and whether serum blocks transfection like KSR, and whether protein-free cryoprotectants are compatible with CAST transfection.

HEK293-GFP cells were grown in standard conditions: 37° C., 5% CO₂, DMEM+10% FBS. The day of transfection, cells were dissociated with TryplExpress (Invitrogen), pelleted by centrifugation, then resuspended in Lonza buffer SF at a concentration of 1E7 cells/ml. 5 ul of cell suspension was added to pre-assembled reaction buffer, and the cells were transfected using Lonza SF-CM-137 protocol. After 72 hours DNA was isolated and PCR for the RELA locus was performed using Primers: 5′-TTCTAGGGAGCAGGTCCTGACT-3′ (SEQ ID NO: 5) and 5′-TCCTTTCCTACAAGCTCGTGGG-3′ (SEQ ID NO: 6). PCR products were purified and subject to Sanger sequencing using primer 5′-TTCTAGGGAGCAGGTCCTGACT-3′. Sequencing results were evaluated using Synthego's ICE tool.

Each transfection reaction contained Ribonuclear Protein complex (RNP) consisting of 20 pMol recombinant Cas9 (Aldevron), complexed with 100 pMol AAVS1 sgRNA sequence GAUCUCCACAUAGGGGCCAG (SEQ ID NO: 7) in 15.5 ul Lonza SF buffer. Mock transfection reactions were performed with Cas9 protein alone in Lonza SF buffer. Each test additive was then added to the 15.5 ul reaction, and total volume was increased to 30 ul by addition of Lonza SF buffer. CryostorCS10 (BioLife Solutions Inc), BamBanker (Fujifilm Wako Chemicals), Recovery Cell Culture Freezing Media (ThermoFisher), Fetal Bovine Serum (FBS) (Sigma Aldrich), Saturated trehalose-dihydrate in water, Glycerol, 33% Polyvinylpyrrolidone Mol wt 40000 (Sigma Aldrich) in water (W/V) were tested at increasing percent of total transfection volume.

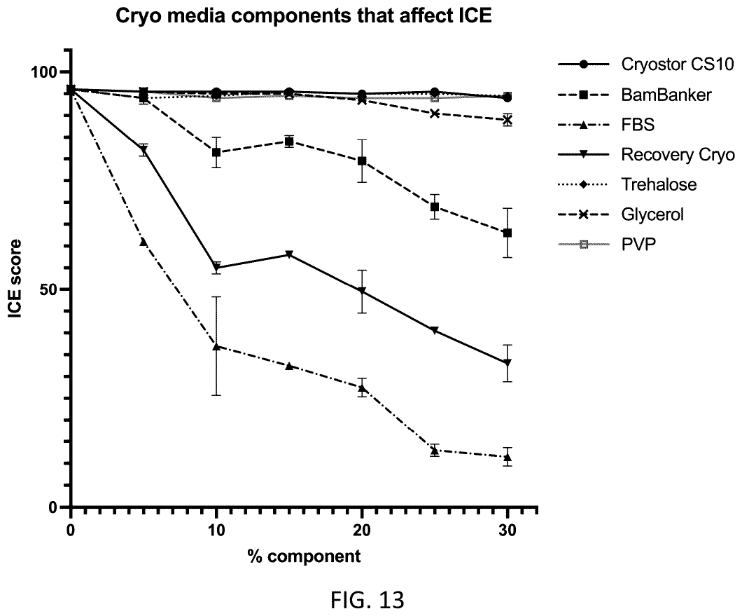

Results are shown in Table 3 and FIG. 13.

TABLE 3

| | 1 | 2 | 3 | 4 | 5 | 6 | 7 | 8 | 9 | 10 | 11 | 12 | |
|---|---|---|---|---|---|---|---|---|---|---|---|---|---|
| Controls | RELA | RELA | RELA | RELA | RELA | | 5% | 10% | 15% | 20% | 25% | 30% | Recovery Cryo |
| | mock | mock | mock | mock | mock | | 5% | 10% | 15% | 20% | 25% | 30% | |
| Cryo store 10 | 5% | 10% | 15% | 20% | 25% | 30% | 5% | 10% | 15% | 20% | 25% | 30% | Saturated Trehalose |
| | 5% | 10% | 15% | 20% | 25% | 30% | 5% | 10% | 15% | 20% | 25% | 30% | |
| Bam Banker | 5% | 10% | 15% | 20% | 25% | 30% | 5% | 10% | 15% | 20% | 25% | 30% | Glycerol |
| | 5% | 10% | 15% | 20% | 25% | 30% | 5% | 10% | 15% | 20% | 25% | 30% | |
| FBS | 5% | 10% | 15% | 20% | 25% | 30% | 5% | 10% | 15% | 20% | 25% | 30% | 33% PVP |
| | 5% | 10% | 15% | 20% | 25% | 30% | 5% | 10% | 15% | 20% | 25% | 30% | |

CryoStorCS10 did not affect editing efficiency. Data showed that small molecules (trehalose, glycerol, PVP) did not affect editing efficiency, while formulations with serum (FBS, CryoRecovery) or undefined protein (BamBanker) reduced editing efficiency. Thus, it was shown that cells frozen in serum-free freezing media can successfully undergo CAST, including when the cells are not washed after being thawed before transfection.

### Example 8

CAST iPSC Germ Layer Formation

iPSCs must be competent to differentiate into all 3 germ layers, endoderm, ectoderm and mesoderm to be considered pluripotent. The following experiments were conducted to determine whether two different iPSC cell lines, PGP1 and r802, maintain the ability to differentiate into different germ layers after CAST editing.

### Cell Culture and Transfection:

iPSC were grown in standard conditions (iMatrix+mTESR+), CAST banked in FreSR-S media (Stemcell Technologies) at 1.9E7 cells/ml. 5 ul of cell suspension was added to pre-assembled reaction buffer, and the cells were nucleofected using Lonza nucleofector. Each transfection reaction contained Ribonuclear Protein complex (RNP) consisted of 20 pMol recombinant Cas9 (Aldevron), complexed with 100 pMol AAVS1 sgRNA sequence ACCCCACAGUGGGGCCACUA (SEQ ID NO: 4) and 40 pMol single strand DNA donor 5′-G*GGTACTTTTATCTGTCCCCTCCACCCCACAGTGGGGCCAATAGGGACAGGATTGG TGACAGAAAAGCCCCATCCTTAGG*C-3′(IDT) (SEQ ID NO: 8) or 100 pMol TCF4 sgRNA sequence ACGGACCCGCAGACGCUCUC (SEQ ID NO: 9) and 40 pMol single strand DNA donor 5′-G*CAGAAGGCAGAGCGTGAGAAGGAGCGGAGGATGGCCAACAATGCCCGAGAGCG TCTGTGGGTCCGTGACATCAACGAGGCTTTCAAAGAGCTCGGCCG*C-3′(IDT) (SEQ ID NO: 10) in 25 ul Lonza P3 buffer.

Transfected cells were plated on iMatrix in mTESR+ supplemented with CEPT inhibitor (R&D systems), and incubated at 32° C. overnight. After 24 hours, media was replaced and cells were incubated at 37° C. for two additional days. iPSC were then dissociated with Accutase (Stemcell Technologies) and expanded on 6 well plates to generate enough cells for differentiation.

Cells were differentiated in 24 well plates using the STEMdiff Trilineage Differentiation kit (Stemcell Technologies) according to the manufacturer's instructions. After differentiation, samples were stored in DNA/RNA shield (ZymoResearch) until total RNA was isolated using Quick-RNA mag bead kits (Zymo Rescarch).

qRT-PCR:

To evaluate gene expression qRT-PCR was performed on each sample in triplicate. To assemble qRT-PCR reactions total RNA was added to wells containing Taqman RNA-to-Ct Mastermix (Applied Biosystems), primer limited human GAPDH endogenous control VIC/MGB probe (Thermo 4326317E), and one of the following: Taqman gene expression assay Hs00751752_s1 (FAM) for Sox 17/endoderm, Taqman gene expression assay Hs01088114_ml (FAM) for Pax6/Ectoderm, Taqman gene expression assay Hs02330376_s1 (FAM) for Hand1/Mesoderm, Taqman gene expression assay Hs04260367_gH (FAM) for OCT4/Pluripotency, Taqman gene expression assay Hs02387400_g1 (FAM) for NANOG/Pluripotency. Samples were run on a BioRad CFX96 Real-Time system using a 2-step qRT-PCR program specified by the RNA-to-Ct kit. Ct values were calculated using default settings in BioRad PCR Maestro software. Fold change compared to undifferentiated controls was calculated using 2(−Delta Delta C(T)) method (PMID 11846609).

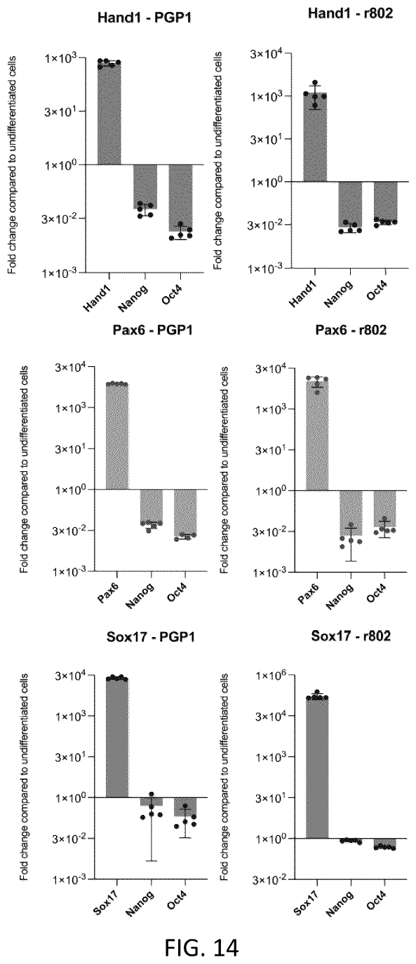

Results are shown in FIG. 14. Both PGP1 and r802 iPSC lines showed induction of the appropriate germ layer marker (PAX6 for Ectoderm, HAND1 for Mesoderm, SOX17 for Endoderm) and loss of pluripotency markers OCT4 and NANOG compared to undifferentiated controls. The data suggested that performing CAST did not compromise the ability of iPSC to give rise to all 3 germ layers.

### Example 9

CAST iPSC Genome Stability

Human iPSCs can change the copy number of certain loci in response to stress. The following experiments were conducted to evaluate whether CAST leads to altered copy number in two iPSC lines, PGP1 and r802.

### Cell Culture and Nucleofection:

iPSC were grown in standard conditions (iMatrix+mTESR+), CAST banked in FreSR-S media (Stemcell Technologies) at 1.9E7 cells/ml. 5 ul of cell suspension was added to pre-assembled reaction buffer, and the cells were nucleofected using Lonza nucleofector. Each transfection reaction contained Ribonuclear Protein complex (RNP) consisted of 20 pMol recombinant Cas9 (Aldevron), complexed with 100 pMol AAVS1 sgRNA sequence ACCCCACAGUGGGGCCACUA (SEQ ID NO: 4) and 40 pMol single strand DNA donor 5′-G*GGTACTTTTATCTGTCCCCTCCACCCCACAGTGGGGCCAATAGGGACAGGATTGG TGACAGAAAAGCCCCATCCTTAGG*C-3′(IDT) (SEQ ID NO: 8) or 100 pMol TCF4 sgRNA sequence ACGGACCCGCAGACGCUCUC (SEQ ID NO: 9) and 40 pMol single strand DNA donor 5′-G*CAGAAGGCAGAGCGTGAGAAGGAGCGGAGGATGGCCAACAATGCCCGAGAGCG TCTGTGGGTCCGTGACATCAACGAGGCTTTCAAAGAGCTCGGCCG*C-3′(IDT) (SEQ ID NO: 10) in 25 ul Lonza P3 buffer.

Transfected cells were plated on iMatrix in mTESR+ supplemented with CEPT inhibitor (R&D systems) and incubated at 32 C overnight. After 24 hours, media was replaced and cells were incubated at 37 C for two additional days.

### DNA Isolation and Copy Number Analysis:

Genomic DNA was isolated using DNeasy Blood and tissue kit (Qiagen) and evaluated using iCS-digital PSC-24 probe kit from stem genomics on BioRad ddPCR equipment according to manufacturer's protocol.

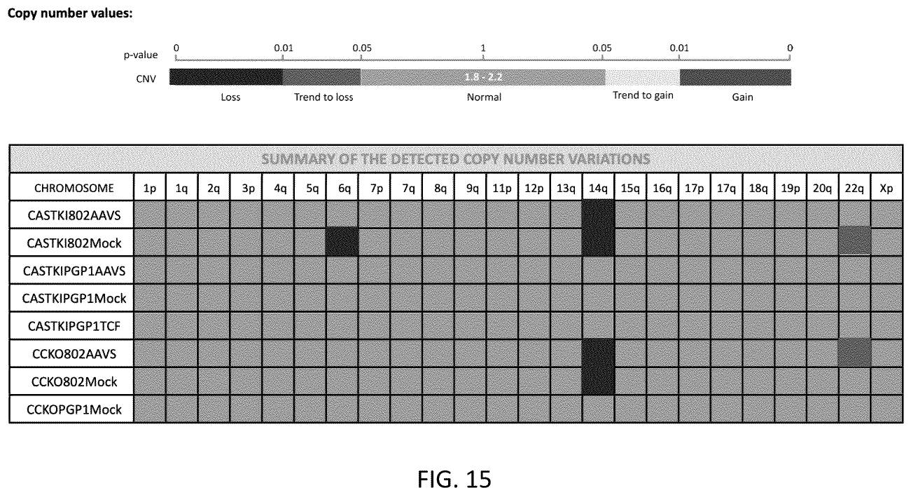

Results are shown in FIG. 15. For all conditions tested no copy number variations were detected in the PGP1 cell line. The R802 cell line showed a loss of 14q present in all samples and the initial bank. Some r802 samples showed ‘trend to loss’ for 22q suggesting heterogeneity in the cell population, but at low levels.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of processing cells comprising:
(i) obtaining cells, wherein the cells are cryopreserved within a freezing media in a cell container;
(ii) thawing the cells in the cell container; and
(iii) contacting the cells with genetic material and a cell manipulation solution to introduce the genetic material into the cells;
wherein the thawed cells are transferred to a separate container before step (iii) or after step (iii).

2. The method of claim 1, wherein the freezing media is serum-free and is optionally also protein-free.

3. The method of claim 2, wherein the freezing media and the cells are transferred to the separate container together.

4. The method of claim 1, further comprising removing at least a portion of the freezing media from the cell container after thawing the cells and prior to transferring without disturbing the cells pelleted at the bottom of the cell container, and optionally resuspending the cells before transferring them.

5. The method of claim 1, wherein the contacting in step (iii) occurs in the separate container, and wherein the separate container comprises (a) the cell manipulation solution, (b) the genetic material, or (c) both the cell manipulation solution and the genetic material, prior to the cells being transferred.

6. The method of claim 1, wherein the cells are not washed or centrifuged after step (ii).

7. The method of claim 1, wherein genetic material comprises one or more polynucleotides.

8. The method of claim 1, wherein the genetic material comprises CRISPR ribonucleoprotein complexes.

9. The method of claim 1, wherein the cells are processed in one or more batches of cells in a multiplicity of cell containers.

10. The method of claim 1, wherein the cells comprise induced pluripotent stem cells (iPSCs).

11. The method of claim 1, further comprising therapeutically administering the processed cells to a subject having a condition, wherein administration of the cells causes a reduction in at least one indication of the condition.

12. The method of claim 1, wherein the time between step (ii) and step (iii) is less than 60 minutes.

13. The method of claim 1, wherein the cell container has a volume of 0.25 mL to 0.5 mL.

14. The method of claim 1, wherein the method is automated after the cells are thawed.

15. The method of claim 1, wherein the cells are frozen prior to step (i) at a concentration of 1e6 to 1e7 cells in 100 μL per cell container.

16. The method of claim 1, further comprising after step (iii) cryopreserving at least a portion of the cells contacted with the genetic material, optionally prior to allowing cell multiplication, to produce cryopreserved cells.

17. The method of claim 16, wherein the cells were contacted in step (iii) with a guide RNA and Cas protein and optionally the cells are cryopreserved before the guide RNA and Cas protein can edit, substantially edit or completely edit the cells.

18. The method of claim 17, wherein the cryopreserved cells are shipped frozen to a different location.

19. The method of claim 1, further comprising after step (iii) placing the cells in a tissue culture plate under conditions that the cells multiply.

20. The method of claim 19, wherein the cells were contacted in step (iii) with a guide RNA and Cas protein and the guide RNA and Cas protein edit the cells when plated.

21. The method of claim 1, further comprising cryopreserving the cells after multiplication, and optionally shipping the cryopreserved multiplied cells to a different location.

22. A method of preparing cells for batch processing of cell editing transfections, the method comprising:
(i) expanding cells to form a cell bank;
(ii) resuspending the cells in a freezing media;
(iii) aliquoting the cells from step (ii) into a multiplicity of cell containers at a concentration of 1e5 to 1e7 cells in 100 μL per cell container, wherein each cell container has a volume of 1 mL or less;
(iv) freezing the cells in the multiplicity of cell containers; and
(v) storing the cells for cell editing transfections.

23. The method of claim 22, wherein the freezing media is serum-free and is optionally also protein-free.

24. A composition comprising cryopreserved cells, wherein the cells comprise a guide RNA and a Cas protein.

25. The composition of claim 24, wherein the cells have not been completely edited.

26. The composition of claim 24, wherein a majority of the cells contain the cellular genome present prior to addition of the guide RNA and Cas protein.

27. The composition of claim 24, further comprising a serum-free freezing media, wherein the media is optionally also protein-free.

28. A method of using the composition of claim 24, further comprising thawing the cells and allowing the cells to be edited by the guide RNA and Cas protein.

29. The method of 28, further comprising therapeutically administering the edited cells to a subject having a condition, wherein administration of the cells causes a reduction in at least one indication of the condition.

30. A method of preparing cryopreserved cells that comprise a Cas protein and a guide RNA, the method comprising:
(i) providing cells;
(ii) contacting the cells with the Cas protein and guide RNA and a cell manipulation solution to introduce the Cas protein and guide RNA into the cells; and
(iii) freezing the cells before the guide RNA and Cas protein can edit, substantially edit or completely edit the cells.

31. The method of claim 30, wherein the cells being provided are cryopreserved and thawed immediately before the contacting step (ii).
